(19) United States
(12) Patent Application Publication
(10) Pub. No.: US 2025/0216394 A1
(43) Pub. Date: Jul. 3, 2025

(54) LIQUID BIOPSY ANALYTES TO DEFINE CANCER STAGES

(71) Applicant: UNIVERSITY OF SOUTHERN CALIFORNIA, Los Angeles, CA (US)

(72) Inventors: Peter KUHN, Los Angeles, CA (US); Anand Ratnakar KOLATKAR, Los Angeles, CA (US); Nicholas MATSUMOTO, Los Angeles, CA (US); Stephanie SHISHIDO, Los Angeles, CA (US); Maryam SETAYESH, Los Angeles, CA (US); Olivia HART, Los Angeles, CA (US); Jeremy Michael MASON, Los Angeles, CA (US)

(73) Assignee: UNIVERSITY OF SOUTHERN CALIFORNIA, Los Angeles, CA (US)

(21) Appl. No.: 18/833,513

(22) PCT Filed: Jan. 30, 2023
(86) PCT No.: PCT/US2023/011889

§ 371 Date: Jul. 26, 2024

Related U.S. Application Data
(60) Provisional application No. 63/304,404, filed on Jan. 28, 2022.

(51) Int. Cl. G01N 33/574; G01N 33/53; G01N 33/543

(52) U.S. Cl. CPC G01N 33/57492; G01N 33/5308; G01N 33/54386; G01N 33/57496; G01N 2333/70503; G01N 2333/70589

(57) ABSTRACT

A biological structure identification system includes an optical imaging system configured to illuminate a liquid biopsy sample for a subject. The liquid biopsy sample has one or more biological structures that are labeled with one or more fluorophores associated with a fluorescence assay for a cancer allowing detection of emitted electromagnetic radiation from the liquid biopsy sample as image data. The system also includes a processing system configured to identify a cancer stage for the subject by determining a predetermined cancer stage profile from a set of predetermined cancer stage profiles to which the subject profile is most similar.

### CROSS-REFERENCE TO RELATED APPLICATIONS

This application claims the benefit of U.S. provisional application Ser. No. 63/304,404 filed Jan. 28, 2022, the disclosure of which is hereby incorporated in its entirety by reference herein.

### TECHNICAL FIELD

In at least one aspect, a system and method for determining cancer stage in a subject is provided.

### SUMMARY

In at least one aspect, a biological structure identification system is provided. The biological structure identification system includes an optical imaging system configured to illuminate a liquid biopsy sample for a subject. The liquid biopsy sample has one or more biological structures that are labeled with one or more fluorophores associated with a fluorescence assay for a cancer allowing detection of emitted electromagnetic radiation from the liquid biopsy sample as image data. The system also includes a processing system configured to:
generate images of the one or more biological structures for the subject from the image data, detect and determine a plurality of features from the images or the image data, and form biological structure identification buckets from the plurality of features, each biological structure identification bucket identifying biological structures that are similar in type;
generating a subject profile of biological structure identification buckets for rare biological structures for the subject;
compare the subject profile with a set of predetermined cancer stage profiles of subjects having the cancer at a plurality of cancer stages; and
identify a cancer stage for the subject by determining a predetermined cancer stage profile from the set of predetermined cancer stage profiles to which the subject profile is most similar.

In another aspect, a method of diagnosing a disease with the biological structure identification system set forth herein is provided. The method includes steps of:
receiving a liquid biopsy sample from a subject comprising biological structures;
preparing a sample comprising a single biological structure layer sample from the liquid biopsy sample, the single biological structure layer sample being a single layer of biological structures;
staining the biological structures of the single layer biological structure sample with a fluorescence assay for a cancer; and
analyzing the sample with a biological structure identification system configured to identify rare biological structures through their fluorescence and morphology and form one or more biological structure identification buckets based on an identified biological structure type, wherein each biological structure identification bucket contains a similar type of biological structures; generating a subject profile of biological structure identification buckets for rare biological structures for the subject;
comparing the subject profile with a set of predetermined cancer stage profiles of subjects having the cancer at a plurality of cancer stages; and
identifying a cancer stage for the subject by determining a predetermined cancer stage profile from the set of predetermined cancer stage profiles to which the subject profile is most similar.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings are of illustrative examples. They do not illustrate all examples. Other examples may be used in addition or instead. Details that may be apparent or unnecessary may be omitted to save space or for more effective illustration. Some examples may be practiced with additional components or steps and/or without all of the components or steps that are illustrated. When the same numeral appears in different drawings, it refers to the same or like components or steps.

For a further understanding of the nature, objects, and advantages of the present disclosure, reference should be had to the following detailed description, read in conjunction with the following drawings, wherein like reference numerals denote like elements and wherein:

FIG. 1-1A. Block illustration of an exemplary biological structure identification system.

FIG. 1-1B. Schematic illustration of an exemplary biological structure identification system.

FIG. 1-2. Illustration of an exemplary identification map comprising identification bucket sets and identification buckets.

FIG. 1-3. Illustration of an exemplary biological structure identification method.

FIG. 1-4. Illustration of an exemplary biological structure identification method.

FIG. 1-5. Illustration of an exemplary method identifying cancer stage.

FIG. 2-1. HDSCA3.0 Workflow Overview. A-C) Blood specimens are collected, processed, and plated onto slides, and undergo immunofluorescent staining. D) Slides are scanned, acquired images are segmented, cellular features are extracted using R and EBImage software, dimensionality reduction analysis is applied to the cells. E) Data processing pipeline allows for rare cell detection, filtering, and classification, and DAPI− event separation for curation of final report.

FIG. 2-2. HDSCA3.0 Rare Event Gallery. A) Images represent two candidate rare events, categorized by marker expression. B) Signal distribution of immunofluorescent markers for channel-classified cells. Designated colors represent each channel-classified group, assigned in A. (LEVs not included due to variation in segmentation)

FIG. 2-3. Enumeration of Circulating Rare Cells. A) Frequency of enumerated rare cells between late-stage, early-stage, and the normal group based on channel classification. B) Comparison of the distribution of rare cells between groups, Kruskal-Wallis H test (one-way ANOVA) performed on all samples. Graphs display total cells per ml. All p values below *0.05 considered statistically significant. C) UMAP rendering of rare cells based on morphometric features. Each designated color represents a classification group marked in A. D) Heatmap illustrating signal intensity of biomarkers on DAPI+|PanCK+ cells detected in late-stage and early-stage BC groups. E) Correlation plot (Pearson correlation) between rare cell categories and LEVs for all samples. Each designated color represents a classification group marked in panel A.

FIG. 2-4. Comparison of Tumor-Associated LEVs A) Frequency of enumerated LEVs between late-stage, early-stage, and the normal group based on channel classification. B) Comparison of the distribution of LEVs between groups, Kruskal-Wallis H test (one-way ANOVA) performed. All p values below *0.05 considered statistically significant. C) Size comparison of LEVs and rare cell events. All sizes represent diameters in micron. Sizes calculated by feature conversion from 100× images. D) Heatmap displaying signal intensity of biomarkers on LEVs and DAPI+PanCK+ cells. E) Scaled frequency plots of rare cells and LEVs in patients.

FIG. 2-5. Clinical Data. A) Comparison of summed LEV levels between differing statuses at last follow-up in early-stage BC. B) Comparison of summed LEV levels between early-stage patients with clinically identified HER2+ and HER2− tumors. Data illustrated in truncated violin plots.

FIG. 2-6. Classification Model. A) On the left, ROC analysis of the random forest model for each target variable class. Curves represent merged prediction from folds. On the right, AUC and F1 score of the corresponding models. B) Confusion matrix of the random forest model on the test set. C) Ranking of the features for classification based on information gain. Each color represents a channel-classified event group detailed in FIG. 2.A.

FIG. 2-7. Supplemental Table.

FIG. 2-8. Supplemental Figure.

FIG. 3-1. Gallery of representative rare events detected by HDSCA3.0 in PB samples collected from BCa patients prior to cystectomy or ND with no known pathology. A-H) rare cells and I) LEVs. A) DAPI only; B) Vim; C) CD45/CD31; D) Vim|CD45/CD31; E) CK|Vim|CD45/CD31; F) CK|CD45/CD31; G) mes.CTC; H) epi.CTC; I) LEVs (top left: CK only; bottom left: CK|Vim|CD45/CD31; top right: CK|CD45/CD31; bottom right: CK|Vim.)] Blue: DAPI, Red: CK, White: Vim, Green: CD45/CD31. Images taken at 100× magnification. Scale bar=10 μm.

FIG. 3-2. Rare event detection using HDSCA3.0 in PB samples collected from BCa patients prior to cystectomy and ND. A) Enumeration and B) frequency of each rare event by channel-type specification. C) Graphical representation of the channel-type rare events/ml between BCa and ND samples ordered by degree of statistical significance. Channel-type specifications that were not statistically significant across the two classifications are highlighted (p>0.05).

FIG. 3-3. Morphometric analysis of individual events detected by HDSCA3.0 in PB samples collected from BCa patients prior to cystectomy. A) tSNE plot of rare cellular events depicting the underlying morphological heterogeneity. Each point represents a single cell and is color coded according to its channel-type classification. B) The same tSNE plot color coded according to a distinct cluster number, as determined by a clustering algorithm. The cells group in multiple clusters spanning across classifications. Visualization of the probability density distributions for select morphometric parameters across channel-type classifications: C) cell area, D) cell eccentricity, E) median CK signal intensity, F) median Vim signal intensity, G) median CD45/CD31 signal intensity.

FIG. 3-4. Patient level classification model using liquid biopsy data. Model statistics for A) NB, SVM, and RF. B) Feature importance from RF.

FIG. 3-5. S1. Site specific liquid biopsy data (Keck; n=25, JHH; n=13, UCSD; n=9, LAC; n=3). A) Bar plot of average counts per patient for each classification and across sites. B) Logarithmic box plot of counts per patient for each classification across sites.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B”. In the case of “only A”, the term also covers the possibility that B is absent, i.e. “only A, but not B”.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

In the examples set forth herein, concentrations, temperature, measurement conditions, and reaction conditions (e.g., pressure, pH, temperature, etc.) can be practiced with plus or minus 50 percent of the values indicated rounded to or truncated to two significant figures of the value provided in the examples. In a refinement, concentrations, temperature, and reaction conditions (e.g., pressure, pH, temperature, etc.) can be practiced with plus or minus 30 percent of the values indicated rounded to or truncated to two significant figures of the value provided in the examples. In another refinement, concentrations, temperature, and reaction conditions (e.g., pressure, pH, temperature, etc.) can be practiced with plus or minus 10 percent of the values indicated rounded to or truncated to two significant figures of the value provided in the examples.

In this disclosure, the indefinite article “a” and phrases “one or more” and “at least one” are synonymous and mean “at least one”.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

The term “event” refers to the detection of an observable imaging signal and in particular to the detection of a fluorescence signal.

The term “feature” refers to any measurable parameter that characterizes an event, image, or image data. For example, features can includes shape parameters, location parameters, texture parameters, and parameters quantifying the fluorescent image.

The term “cluster” refers to a group of similar data points. In a refinement, data points can be grouped together based on the proximity of the data points to a measure of central tendency of the cluster. For example, the measure of central tendency may be the arithmetic mean of the cluster. In such an example, the data points are joined together based on their proximity to the average value in the cluster. (e.g., hierarchical clustering).

The term “similar” when referring to data points means that the data points can be placed in the same cluster. That is, similar data points can be placed or included within the same cluster after a clustering analysis. In a refinement, a cell (or other biological structure) is similar to another cell (or other biological structure) if the cell (or other biological structure) belongs in the same cluster after cluster analysis (hierarchical clustering), which is an algorithm that groups similar objects into groups. OCULAR applies a Principal Component Analysis onto the high dimensional dataset and then undergoes hierarchical clustering on the distance matrix of the PCA dataset. The output of the hierarchical algorithm determines which cells (or other biological structures) are similar to another by determining which cluster each cell belongs in. In another refinement, a set of cellular features (e.g., biological structures) is similar to another set of cellular features if the distance of the principal components between those sets is within the 1 percentile of all distances found in the distance matrix of a large dataset, which includes those sets, that underwent PCA.

The term “imaging event” means imaging structures that are defined by imaging parameters collected by the imaging system without applying biological context/relevance.

The term “profile of biological structure identification buckets” means a predetermine collection of biological structure identification buckets. Therefore, the user or an algorithm can select a plurality of biological structure identification buckets from which profiles are formed. Profiles for a characterizing a cancer stage are a specific collection of biological structure identification buckets that are common to a cohort of human samples in a specific cancer stage. A profile from a given human sample can be computationally/mathematically compared to a reference cohort-determined cancer stage profiles to determine the similarity of the given sample to the reference profiles.

The term “computing device” refers generally to any device that can perform at least one function, including communicating with another computing device.

The term “computing device” refers generally to any device that can perform at least one function, including communicating with another computing device.

When a computer or other computing device is described as performing an action or method step, it is understood that the computer or other computing device are operable to and/or configured to perform the action or method step typically by executing one or more lines of source code. The actions or method steps can be encoded onto non-transitory memory (e.g., hard drives, optical drive, flash drives, and the like).

The term “configured to or operable to” means that the processing circuitry (e.g., a computer or computing device) is configured or adapted to perform one or more of the actions set forth herein, by software configuration and/or hardware configuration. The terms “configured to” and “operable to” can be used interchangeably.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

Throughout this application, where publications, patents, or published patent applications are referenced, the disclosures of these publications, patents, or published patent applications in their entireties are hereby incorporated by reference into this application to more fully describe the state of the art to which this invention pertains.

### Abbreviations and Acronyms

+: positive, when associated with a marker (e.g., CD31+, CD45+, CK+, vimentin+) or a chemical molecule (e.g., DAPI+), the cell or biological formation expresses this marker or a chemical molecule.

−: negative, when associated with a marker (e.g., CD31−, CD45−, CK−, vimentin−) or a chemical molecule (e.g., DAPI−), the cell or biological formation does not express this marker or a chemical molecule.

CD31: platelet endothelial cell adhesion molecule-1.

CD45: leukocyte-common antigen.

CPU: central processing unit.

CTC: circulating tumor cell

CRE: circulating rare event

CK: cytokeratin.

DAPI: 4′,6-diamidino-2-phenylindole.

HDSCA: High Definition Single Cell Assay.

OCULAR: Outlier Clustering Unsupervised Learning Automated Report.

PCA: principal component analysis.

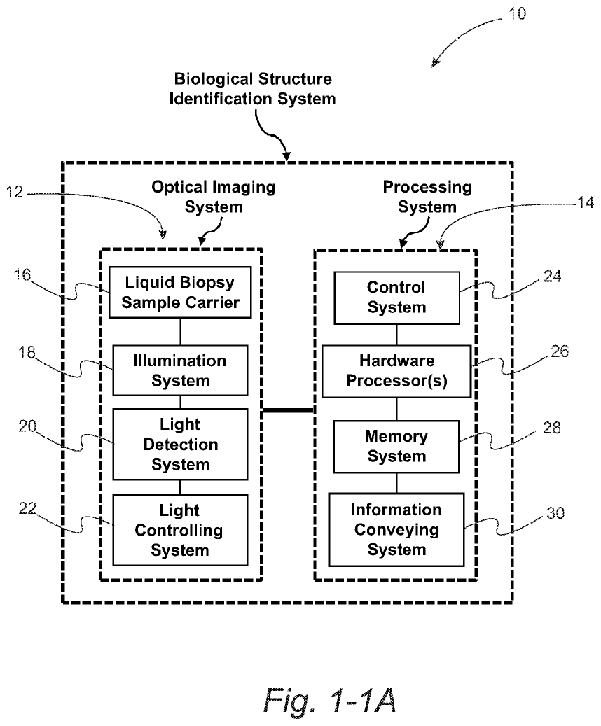

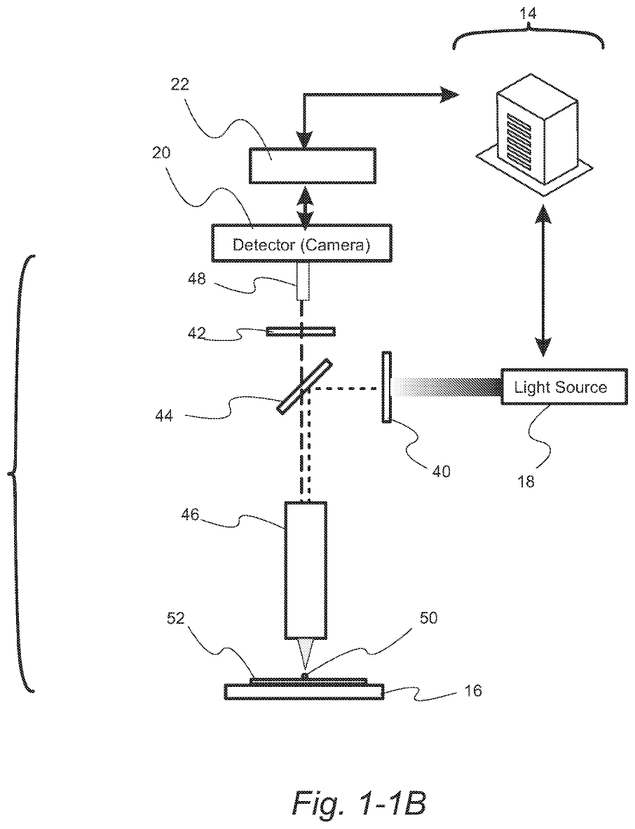

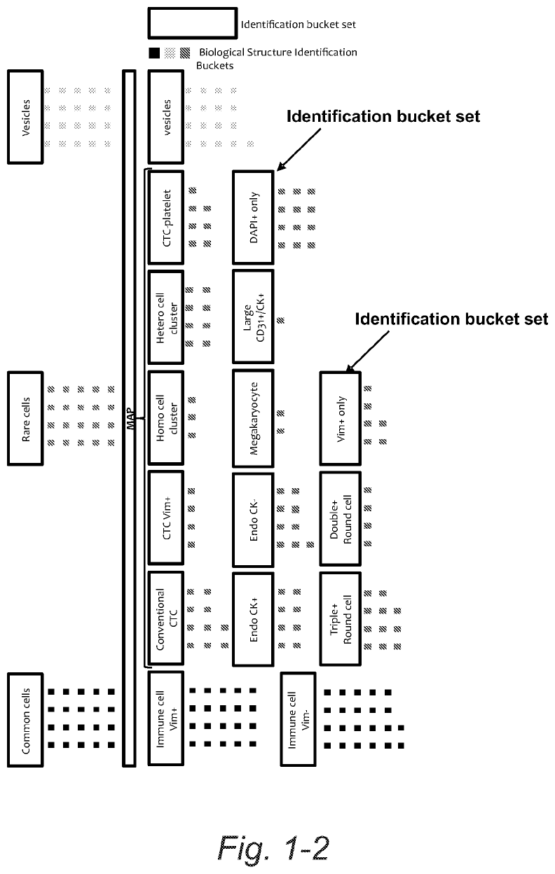

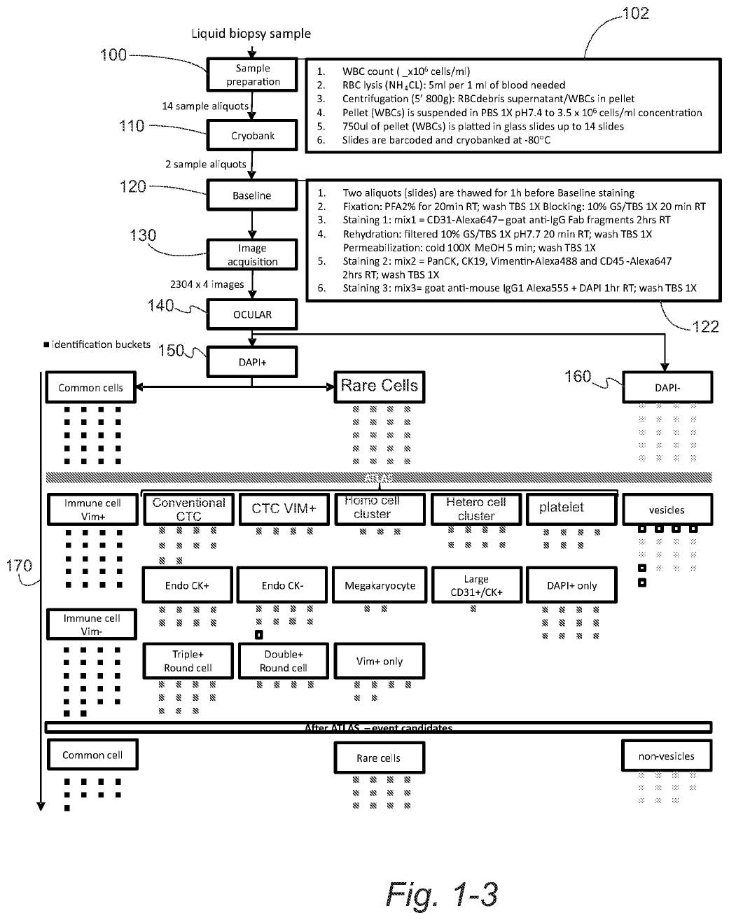

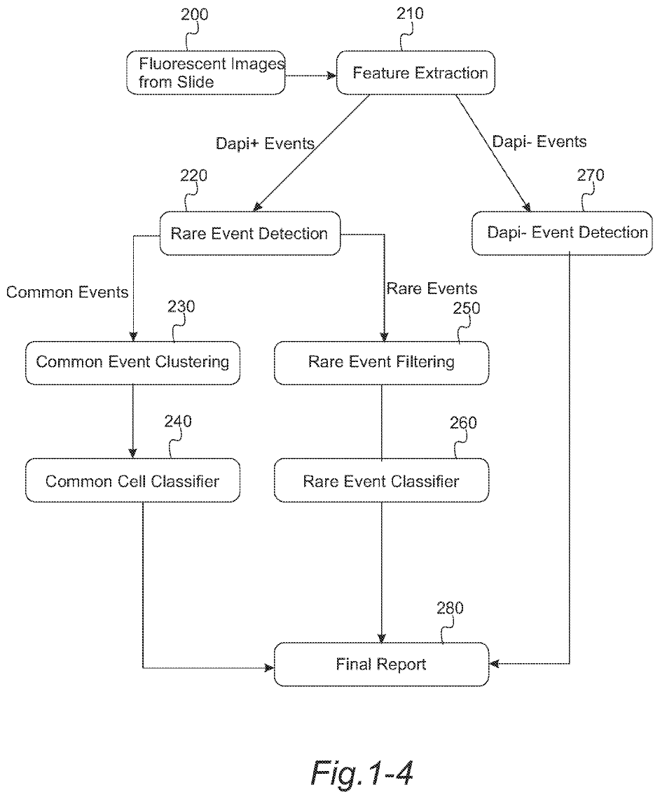

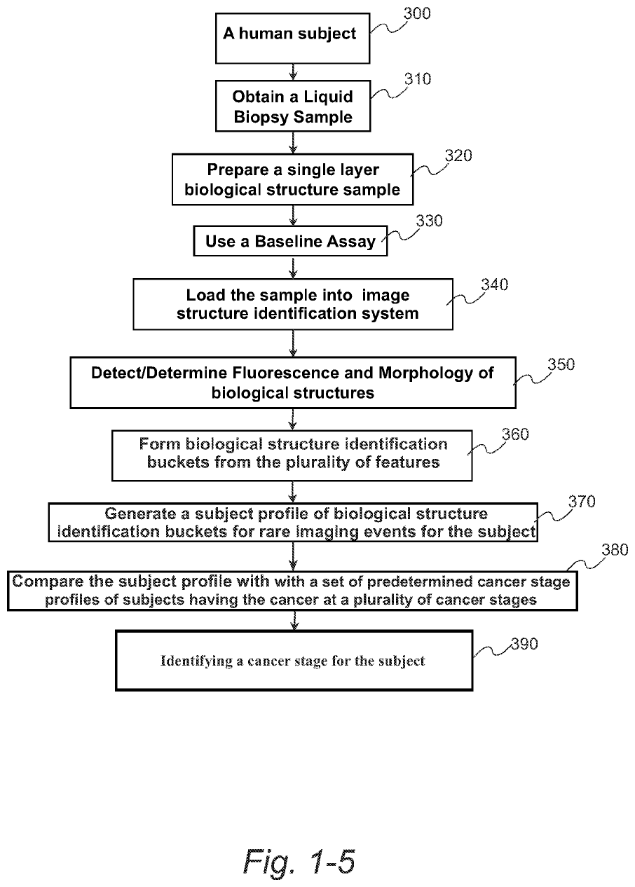

Referring to FIGS. 1-1A and 1-1B, an example of a system for identification for evaluating a subject with respect to a disease state is provided. Biological structure identification system 10 includes an optical imaging system 12 and a processing system 14. The liquid biopsy sample typically includes one or more biological structures that may be labeled with one or more fluorophores. Characteristically, optical imaging system 12 is configured to illuminate a liquid biopsy sample having one or more biological structures that are labeled with one or more fluorophores associated with a fluorescence assay for a cancer allowing detection of emitted electromagnetic radiation from the liquid biopsy sample as image data. Processing system 14 is configured to
generate images of the one or more biological structures for the subject from the image data, detect and determine a plurality of features from the images or the image data, and form biological structure identification buckets from the plurality of features, each biological structure identification bucket identifying biological structures that are similar in type;
generate a subject profile of biological structure identification buckets for rare biological structures for the subject;
compare the subject profile with a set of predetermined cancer stage profiles of subjects having the cancer at a plurality of cancer stages; and
identifying a cancer stage for the subject by determining a predetermined cancer stage profile from the set of predetermined cancer stage profiles to which the subject profile is most similar. In a refinement, the processing system is further configured to:
detect and determine morphology of each biological structure using each image;
identify type of each biological structure from the plurality of features;
form the biological structure identification bucket plurality of features; and
form a set of identification buckets based on identification buckets. Typically, processing system 14 is or includes a computing device.

In a variation, the rare imaging events are observed as rare biological structures.

In another variation, one or more biological structures include simultaneously identified multiple biological structures.

In another variation, the rare biological structures are observed as rare imaging events in the imaging data.

Still referring to FIGS. 1-1A and 1-1B, the optical imaging system 12 can include a liquid biopsy sample carrier 16 suitable for supporting the liquid biopsy sample for the identification of the biological structure(s); an illumination system 18 capable of illuminating the liquid biopsy sample at a specific wavelength or wavelengths that can be absorbed by the fluorophore; a light detection system 20 configured to detect and determine an intensity and a wavelength of fluorescence emitted by the fluorophore; and a light controlling system 22. The light controlling system 22 can be configured to allow detection of emitted electromagnetic radiation from the liquid biopsy sample; allow detection of electromagnetic radiation scattered by, reflected by, and/or transmitted through the liquid biopsy sample; and guide electromagnetic radiation from the illumination system to the liquid biopsy sample, and from the liquid biopsy sample to the light detection system. Also depicted if FIG. 1-1B, optical system 22 may include an optical component selected from the group consisting of an excitation filter 40, an emission filter 42, a (dichroic) mirror 44, a lens 46, an optical fiber 48, and combinations thereof. FIG. 1-1B also shows specimen 50 positioned on glass slide 52. In a refinement, light from an illumination system 18 (e.g., a laser light source) is passed through excitation filter 40 and then to dichroic mirror 44 which directs the excitation light through lens 46 (e.g., an objective lens). Lens 46 focuses the light onto specimen 50. The resulting emitted or scattered light passes through lens 46, dichroic mirror 44, and emission filter 42. The fluorescent light is then detected by light detection system 20 optionally through fiberoptic 48.

Still referring to FIG. 1-1, the processing system 14 may include a control system 24, a hardware processor 26 (e.g., CPU), a memory system 28, and an information conveying system 30. Processing system 14 will execute the analysis step via hardware processor 26. Control system 24 is the executing software components that a user uses to control and interact with the optical imaging system 12 and to initiate analysis and image construction from the image data received from the optical imaging system. The information conveying system 30 is configured to convey to a user information comprising an information related to types of the biological structures present in the liquid biopsy sample, the biological structure identification buckets, the disease maps, the disease atlases, or a combination thereof. Control system 24 and information conveying system 30 function via program codes executing on hardware processor 26 and via software and data stored in memory system 28

In a variation, the biological structure identification system 10 is configured to receive a liquid biopsy sample by using the liquid biopsy sample carrier 16 and illuminate the liquid biopsy sample with an electromagnetic radiation from illumination system 18 that has a specific wavelength or wavelengths that can be absorbed by the fluorophore. Light detection system 20 is configured to detect and determine an intensity and a wavelength of fluorescence emitted by the fluorophore with light detection system 20 or produce input data for these characteristics so that they can be determined by processing system 12.

Processing system 14 is configured to generate an image of the biological structure(s) from image data received from light detection system 20; detect and determine a morphology of each biological structure from the image and/or the image data using the plurality of features; identify the type of each biological structure based on the features defined herein (which can determine a specific morphology) of each biological structure; form a biological structure identification buckets (“identification bucket”) based on the identified biological structure type such that each biological structure identification bucket contains the biological structure(s) that are similar in type and in particular cells containing such biological structure(s); and optionally, form a set of identification buckets (“identification bucket set”) based on the identification buckets. In a particularly useful variation, the biological structures are cells that a placed in the identification buckets and in the identification bucket set. In this context, “placed in” means that an association between the cells (or other biological structures) and the identification buckets and in the identification bucket set is saved in computer readable form as set forth below. In the context of the present embodiment, each biological structure identification bucket identifying imaging structures that are similar in type.

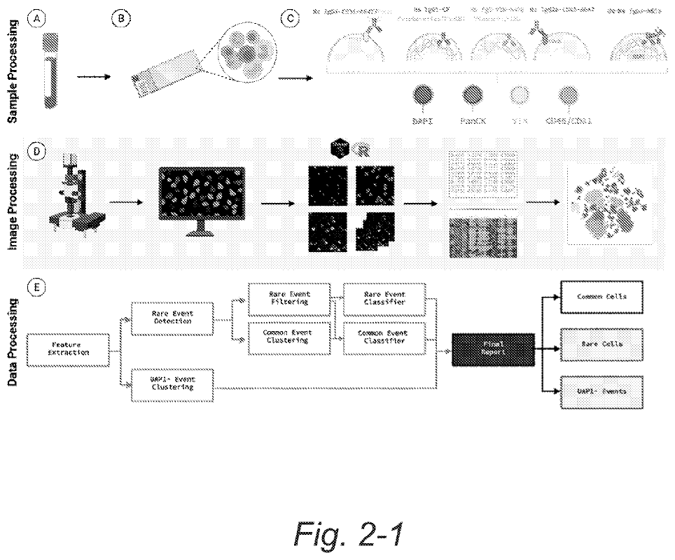

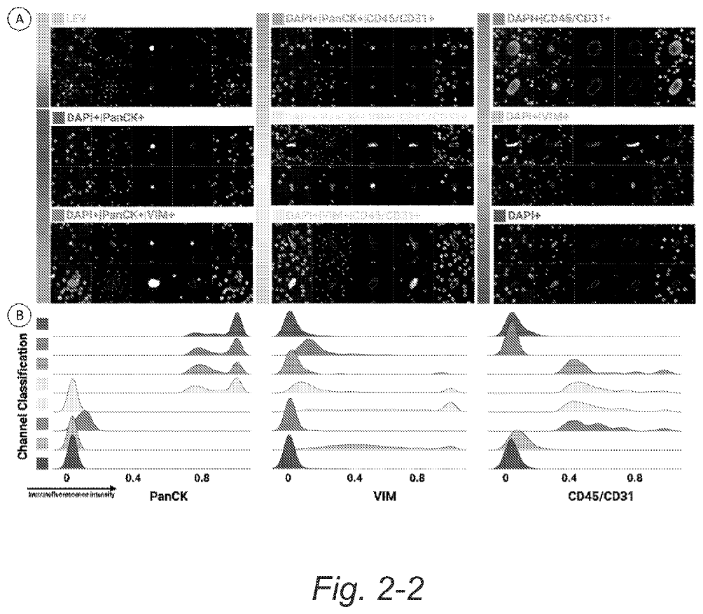

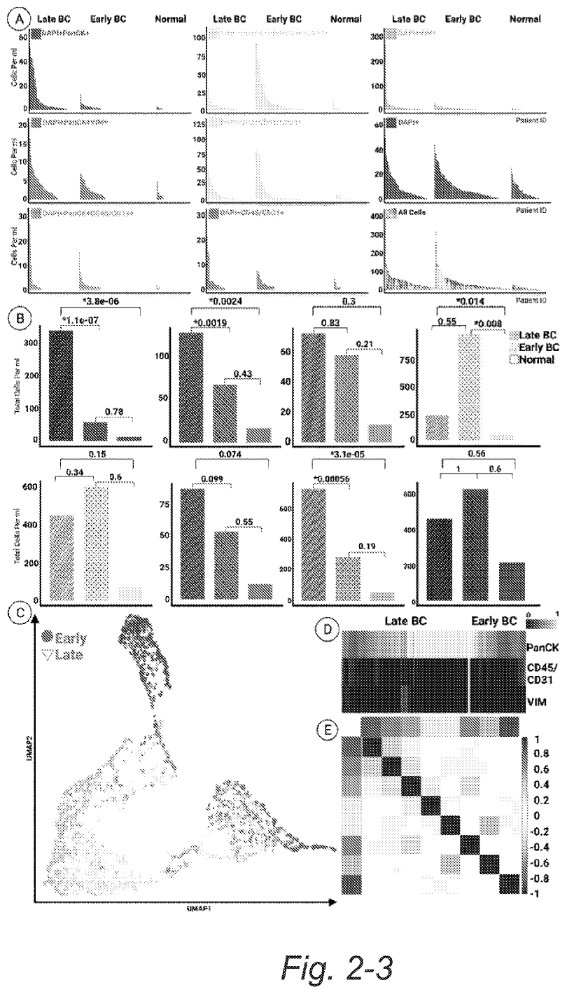

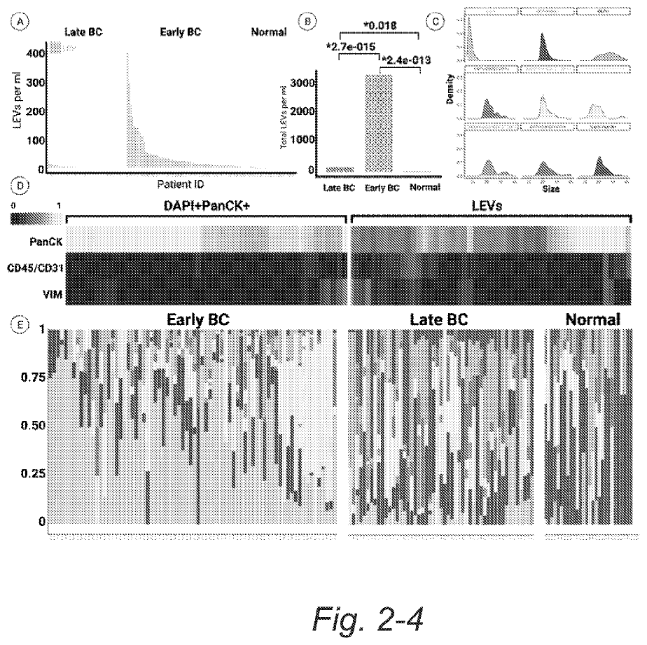

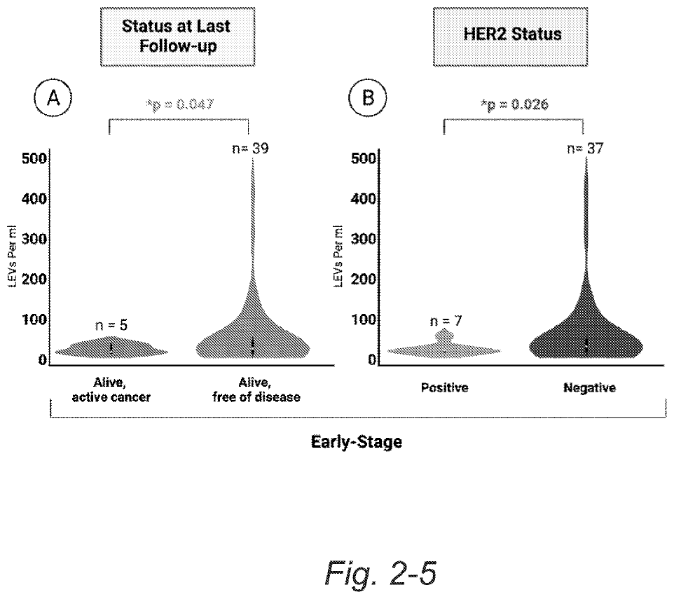

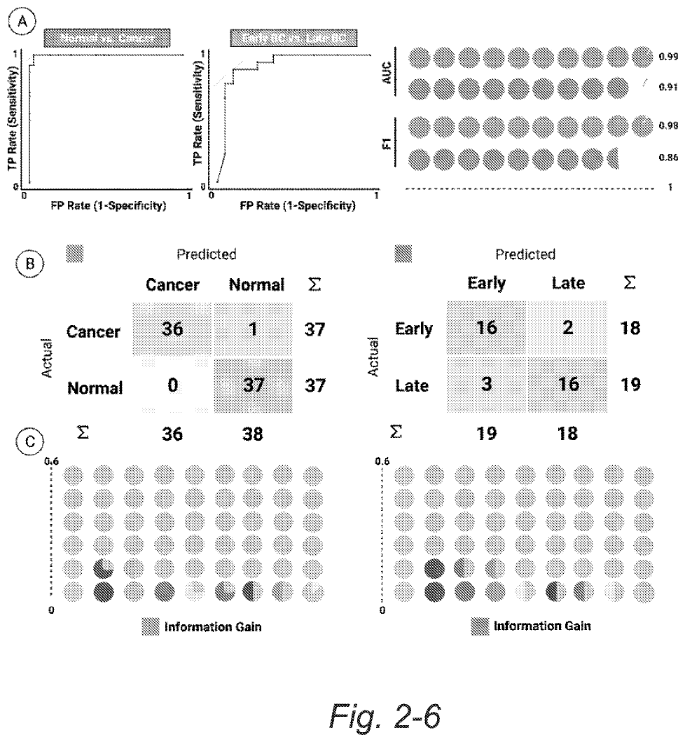

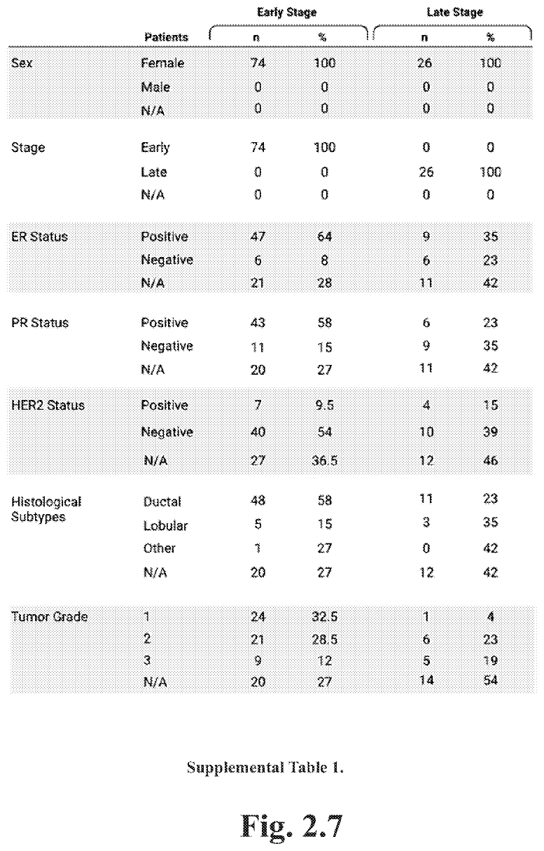

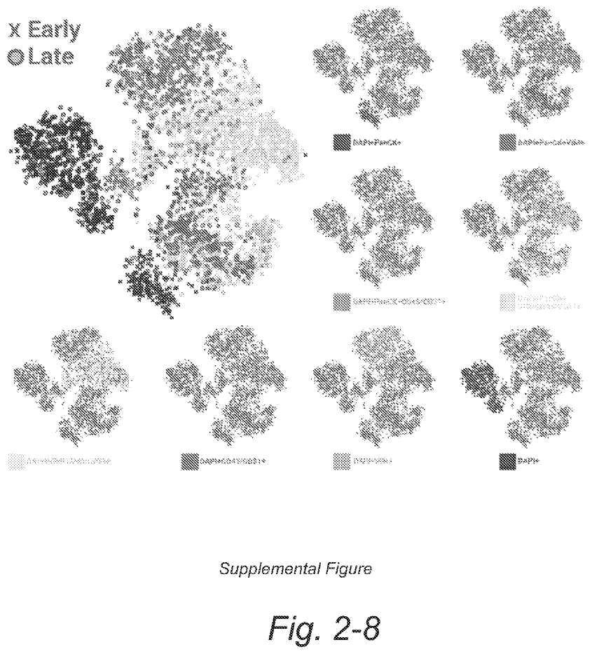

FIG. 1-2 illustrates exemplary identification buckets and identification bucket sets. From this figure, identification bucket sets are constructed from a plurality of identification buckets. In this figure the identification buckets are identified by smaller squares that are color coded to provide a representation of the number of cells in a bucket. For example, the black buckets under common cells represent a high number of cells (or biological structures) in these buckets. The buckets and bucket sets can be associated with any label that is convenient for the user. It should be appreciated that the identification buckets and identification bucket sets are typically stored in a computer readable medium and in particular a non-transitory computer readable medium (e.g., random access memory, CDROM, DVD, hard drive, etc.). In a refinement, identification buckets and identification bucket sets are stored in a computer readable medium as a data structure with relationship between the stored values. Examples of data structures that can be used include, but are not limited to arrays, linked lists, records, a graph, a tree data structure (e.g., a binary tree), a data frame, a database (e.g., a relational database), and combinations thereof.

In a variation, processing system 14 is further configured to form a disease map based on information related to the biological structure identification bucket set(s), relate the disease map to a specific disease and disease stage, and label the disease map according to an identified related specific disease and disease stage.

In some aspects, the morphology of the biological structure may be determined by using at least one feature extracted from the image or image data. Typically, the image data will include features (e.g., parameters) of the fluorescent light emitted from the sample. These features can be extracted from the generated image or the image data by using know software packages such as the EBImage which is an open source R package distributed as part of the Bioconductor project. The morphology of the biological structure may be determined by using at least 10 features, at least 100 features, at least 500 features, or at least 1,000 features extracted from the image or image data. Features can include shape parameters, location parameters, texture parameters, and parameters quantifying the fluorescent image (e.g., specific fluorescence wavelength(s), fluorescence signal intensity, etc.). The feature may be related to size, shape, texture and structure of the biological structure's morphology. In some variations, an image mask is deployed limiting the observable image area to regions encompassed by the mask. Table 1 provides non-limiting examples of features that can be used in the analysis. Any combination of the features in Table 1 can be used.

TABLE 1
List of parameters (i.e., features) pulled
from the mask and image data with the mask.

| | | | Haralick Parameters |
|---|---|---|---|
| | Moment parameters | Shape parameters | using the Gray |
| | of Cell Mask | of Cell Mask | Level Co-occurrence |
| Basic parameters | and Nucleus mask | and Nucleus mask | Matrix |
| Mean Intensity | cell x position | Area | Angular second |
| Standard Deviation | within ROI | perimeter | moment |
| of pixel Intensity | cell y position | Mean radius | contrast |
| Median absolute | within ROI | Standard deviation | Correlation |
| intensity | Major axis | of radius of Mask | Variance |
| 1%, percentile | Eccentricity | Minimum radius | Inverse difference |
| intensity | Theta | Maximum radius | moment |
| 5%, percentile | | | Sum average |
| intensity | | | Sum variance |
| 5O % percentile | | | Sum entropy |
| intensity | | | Entropy |
| 95%. percentile | | | Variance difference |
| intensity | | | Entropy Difference |
| 99% percentile | | | Correlation measures |
| intensity | | | |

In some refinements, the identification bucket may be a specific repository (e.g., classification) where information related to a specific biological structure(s) identified in a liquid biopsy sample is stored, wherein the specific biological structures may have substantially similar properties, including substantially similar morphologies and substantially similar marker profiles. The information related to the specific biological structure may be any information related to the biological structure, including the identification bucket's label, number of the specific biological structures identified in a given portion of the liquid biopsy sample analyzed, properties associated with the specific biological structure, information related to the liquid biopsy sample, the like, or a combination thereof. This information related to the specific biological structure(s) may be stored in any convenient manner. For example, the information related to the specific identification bucket may be stored in the memory system. In some refinement, the bucket is a cluster as described below.

In some aspects, at least a subset of the biological structure can a structure with a membrane, a protein, DNA, RNA, or a combination thereof. The structure with a membrane may be a cell, a vesicle, or a combination thereof. The vesicle may be an oncosome. The oncosome may have a characteristic size (e.g. characteristic length or characteristic diameter) equal to or larger than one micrometer. The oncosome may have a characteristic size (e.g. characteristic length or characteristic diameter) larger than an exosome.

In other aspects, the liquid biopsy sample may be a non-solid biological sample. The liquid biopsy sample may be a body fluid sample. The liquid biopsy sample may include a blood sample, a bone marrow sample, a peritoneal fluid sample, a urine sample, a saliva sample, a vaginal fluid sample, a semen sample, a tear sample, a mucus sample, an aqueous humor sample, cerebrospinal fluid (CSF) sample, or a combination thereof. The liquid biopsy sample may include a blood sample. The liquid biopsy sample may include common immune cells and rare biological structures.

In still other aspects, the rare biological structures may include cancer cells that have cancer genomic profiles and/or cancer protein markers; tumor microenvironment cells that leak into circulation, wherein these cells comprise epithelial cells, endothelial cells, mesenchymal cells, other stromal cells, cells that are in various transitional states, or a mixture thereof; immune cells that are responding to the tumor itself or cancer treatment; vesicles, or a mixture thereof. The rare biological structures may include conventional circulating tumor cells, which are CK+, vimentin−, CD31− and CD45−; circulating tumor cells, which are CK+, CD31−, CD45−, and vimentin+, and wherein tumor cells may putatively in epithelial to mesenchymal transition; tumor cells, which are CK+, and coated with platelets, which are CD31+; endothelial cells, which are CD31+, vimentin+, and CK−; endothelial cells, which are CD31+, vimentin+ and CK+; megakaryocytes, which are CD31+ and vimentin−, wherein megakaryocytes may comprise large cells containing a single, large, multi-lobulated, polyploidy nucleus responsible for the production of blood thrombocytes platelets; large cells, which are CD31+, and cytokeratins, which are CK+, wherein these large cells may be present in the liquid biopsy samples obtained from a bone marrow; large cells, which are CD31+ and CK+, wherein these large cells may be present in liquid biopsy samples obtained from a bone marrow; cells, which are DAPI+ and vimentin+; round cells, which are CD45+ and CK+; round cells, which are CD45+, vimentin+, CD45+, and CK+; clusters of cells (“cell cluster’) comprising at least two cells, wherein the cells are same type of cells and/or different types of cells; cells, which are DAPI+, CD45−, CD31−, and CK−; immune cells, which are CD45+ and vimentin−; immune cells, which are CD45+ and vimentin+ (type III intermediate filament protein), extra-cellular vesicles, or a mixture thereof.

In some aspects, the liquid biopsy sample may include common biological structures and rare biological structures. A total number of biological structures is a sum of the number of common biological structures and the number of rare biological structures. Characteristically, the fraction of the rare biological structures are equal to or less than 10%, 5%, 1%, 0.1%, or 0.01% of the total number of biological structures.

In a refinement, the optical imaging system includes a fluorescence imaging system, a brightfield imaging system, or a combination thereof. The optical imaging system may include a fluorescence microscope, a brightfield microscope, or a combination thereof.

In some aspects, the emitted electromagnetic radiation may be a fluorescent radiation.

In some aspects, the biological structure identification system includes at least one fluorescence channel. The number of fluorescence channels may be in the range of 1 to 10 fluorescence channels, or in the range of 4 to 7 fluorescence channels. In a refinement, the number of fluorescence channels may be only four. These four fluorescence channels may be a first fluorescence channel configured for detection useful for nuclear segmentation and characterization; a second fluorescence channel configured to detect a cytokeratin (CK) for its epithelial-like phenotype; a third fluorescence channel configured to detect a vimentin for its endothelial/mesenchymal-like phenotype; and a fourth fluorescence channel configured to detect both a CD31 for its endothelial-like phenotype, and a CD45 for its immune cell phenotype. These four fluorescence channels may be a first fluorescence channel configured for detection of fluorescence emission at a blue color wavelength region; a second fluorescence channel configured for detection of fluorescence emission at a red color wavelength region; a third fluorescence channel configured for detection of fluorescence emission at an orange color wavelength region; and a fourth fluorescence channel configured for detection of fluorescence emission at a green color wavelength region. For example, these for regions can be defined by an emission filter centered at 455 nm with a bandwidth of 50 nm for blue color wavelengths, an emission filter centered at 525 nm with a bandwidth of 36 nm for green color wavelengths, an emission filter centered at 605 nm with a bandwidth of 52 nm for orange color wavelengths, and an emission filter centered at 705 nm with a bandwidth of 72 nm for red color wavelengths. The first immunofluorescence channel may be configured to detect 4′,6-diamidino-2-phenylindole (DAPI) for nuclear segmentation and characterization.

In some aspects, the systems of this disclosure may be configured to identify endothelial cells and immune cells from the features and/or the morphology of the endothelial cells and the immune cells determined from the features. In particular, the system can be configured to identify the endothelial cells and the immune cells from the features (and/or morphology of the endothelial cells and the immune cells determined from the features), and to differentiate the endothelial cells from the immune cells. The endothelial cells may have more elongated morphologies as compared to the immune cells, and the immune cells may have more round morphologies as compared to the endothelial cells. In a refinement, such morphologies are determined from the features as described herein.

In some aspects, the liquid biopsy sample is obtained from a diseased human. For example, the liquid biopsy sample may be obtained from a human afflicted with a cancer.

In some refinements, the biological structure identification system is further configured to form a disease map based on information related to the identification bucket set(s), relate this disease map to a specific disease and disease stage, and label this disease map according to the related specific disease and its stage. The biological structure identification system may further be configured to store a disease map based on information related to the identification bucket set(s) and labeled by a disease type and the disease stage, and wherein the disease may cause formation of the biological structures forming said identification bucket set(s). The biological structure identification system is configured to form disease maps of at least two different types of diseases and stages of each disease.

The biological structure identification system may further be configured to form a disease atlas (“ATLAS) of disease maps based on the disease maps of different disease types and their stages. In this regard, the atlas built by using the trillions of cellular data, performing a PCA on the dataset and then selecting the cells that would create a dataset that would have a non-overlapping region in that PCA dataspace. Each cell would represent a certain region of that space such that any subsequently scanned cell would necessarily belong to a cell in the atlas. A cell would be assigned an ATLAS cell ID by applying the ATLAS PCA transform and finding the closest ATLAS cell. For example, identifying clusters into which a cell from a patient belongs can be used to assist in cancer identification and prognosis. In this context, “belong” means that the cell (or other biological structure) has feature values representative of the cluster (e.g., within the parameter or feature boundaries of the cluster). In some refinements, the atlas and/or the disease maps in atlas include metadata such as patients' identification, clinical parameters, image parameters and the like. The atlas and/or the disease maps can include this data for each cell (or other biological structures) contained therein. In a refinement, the disease atlas is stored in a computer readable medium and in particular a non-transitory computer readable medium (e.g., random access memory, CDROM, DVD, hard drive, etc.). In a refinement, the disease atlas is stored in a computer readable medium as a data structure with relationship between the stored values. Examples of data structures that can be used include, but are not limited to arrays, linked lists, records, a graph, a tree data structure (e.g., a binary tree), a database (e.g., a relational database), and combinations thereof. In a refinement, the disease atlas is stored as a database and in particular, a relational database that can be queried.

In some refinements, the biological structure identification system is further configured to diagnose the disease type and its stage based on the received liquid biopsy sample from a human afflicted with a disease. The biological structure identification system may further be configured to diagnose the disease type and its stage based on a liquid biopsy sample received from a human afflicted with a disease by comparing the disease map formed for the received liquid biopsy sample with the disease maps of the disease atlas stored in the biological structure identification system prior to receiving the liquid biopsy sample.

In a variation, an immunofluorescence assay for analyzing a liquid biopsy sample is provided. This assay may include antibodies against cytokeratin (CK), vimentin, CD31 and CD45. In a refinement, at least a subset of the antibodies against cytokeratin (CK), vimentin, CD31 and CD45 are labeled with a fluorophore. In the Baseline assay, each of cytokeratin (CK) and vimentin are independently labeled with a fluorophore while one or both of CD31 and CD45 are labeled with a fluorophore. Examples of fluorophores include but are not limited to, DAPI and Hoechst 33342 and 33258 (as nuclear dyes), Alexa Fluor 488 (for Vimentin), Alexa Fluor 555 (for cytokeratin), Alexa Fluor 647 (for CD31/CD45), and the like.

In a variation, a method of analyzing a liquid biopsy sample is provided. This method may include having a liquid biopsy sample comprising biological structures; preparing a sample comprising a single layer of biological structures (“single layer biological structure sample”) by using the liquid biopsy sample; staining the biological structures of the single layer biological structure sample with the fluorescent assay(s) set forth herein (having four fluorescent dyes) or any fluorescent assay; using the biological structure identification system (s) of this disclosure; identifying the rare biological structures through their fluorescence and morphology; and forming a biological structure identification bucket based on the identified biological structure type, wherein each biological structure identification bucket may contain a similar type of biological structures. FIGS. 1-3 and 1-4 provide exemplary liquid biopsy sample analysis methods.

Referring to FIG. 1-3, a flow chart of the sample analyst method is provided. As depicted in Box 100 the liquid biopsy sample is processed in accordance to a predetermined protocol. Box 102 provides an example of such a protocol. In a refinement as depicted in box 110, sample aliquots are optionally stored in a cryobank. In the next step (Box 120), a fluorescence assay is used to stain the liquid biopsy sample (e.g., an immunofluorescence assay such as the Baseline assay (see below) or any fluorescence assay). Box 122 provides a specific example of this processing. The image data is then acquired as shown in box 130. In box 140, the acquired image data is then analyzed. The Ocular analysis protocols described below in more detail can be applied for this analysis. As part of the analysis, the image data can then be segregated into DAPI+ (box 150) or DAPI− (Box 160) regions. Each region is subjected to cluster analysis as set forth below to identify buckets for classifying the cells.

Referring to FIG. 1-4, a flow chart of an exemplary liquid biopsy sample analysis method is provided. Typically, this analysis is implemented by processing system 14 or another computing device. As depicted in box 200, fluorescent images are received as an input to processing system 14. As part of the analysis nuclear and/or cell masks are generated and features extracted (e.g., over 700 features pulled from the 4 fluorescent images). As depicted by box 210, rare event detection proceeds as follows. For each region on the slide, the data for each event will undergo dimensional reduction, and then will be hierarchical clustered into multiple groups. The number of clusters are determined by how large the dataset is. For each frame (region) on the slide, we divide the total number of cells within the region by 30 and round the number to an integer which is the number of clusters that the multidimensional data would cluster into. Rare events are defined as 1) events within the smallest population clusters and 2. events with the clusters that are most deviant from the median value of all features from all events. Rarity within a region on the slide is defined via cluster analysis. After the region undergoes feature extraction and hierarchical clustering on principal components, the clusters are sorted by 2 quantifiable measures: 1) population size in ascending order and 2) the Euclidean distance of the cluster's mean feature of all cells within the cluster to the median feature of all cells of the whole region in descending order. Clusters that are towards the top of these two lists are considered rarer than the clusters towards the bottom of the lists. In a refinement, rare events are below a predetermined rarity threshold. In a further refinement, the rare events are below a rarity threshold of 1.5%. The rarity threshold is the percentage of cells within a region on the slide of which the algorithm will define as rare. The rarity threshold is applied after sorting the clusters with the above measures. The rarity threshold is a value that can be passed into the algorithm by defining it as an argument. Separately with the two sorted lists of clusters, the algorithm will add up the rarer clusters until the total number of cells cross the rarity threshold. After performing this step with the two rare lists, the algorithm returns the unique list of cells that are within such clusters. These are the rare cell candidates within the region of the slide. Rarity within the slide is performed by a filtering method of using common cell clusters throughout all the regions of the slide and removing all rare cell candidates that are similar to any such common cell cluster. Similarity in this case is determined by the PCA dataset of both rare cell candidates and common cell cluster features. After performing a distance matrix of the combined PCA dataset, the value of the 1 percentile of all distances found in the matrix will be the maximum distance necessary to be considered similar. If a rare cell candidate is within that value of any common cell cluster, that rare cell candidate will not be labeled as rare and will add onto the respective common cell cluster that was most similar to that rare cell candidate. Each region will collect up to a certain user defined percentage of rarity of the total cells within the region. The rare event features are individually collected and sent through the rare event pipelines as rare event candidates. Each rare event includes the position of the event on the slide. In another refinement, rare biological structures (e.g., cells) are biological structures that are identified as being below a predetermined percentage of the total amount of biological structures identified. In a refinement, this predetermined percentage is in increasing order of preference, 5%, 4%, 3%, 2%, 1.5% or 1% of the total number of identified biological structures. The common events are aggregated into their respective common event cluster as a mean of the features of the events within the cluster. They are sent through the common event pipeline in this aggregated form.

As depicted by box 230, common event clustering is analyzed as follows. Since the common clusters from the previous step are determined by a single region on the slide, each common cluster is then clustered together by their similarity. The sum of those events are preserved as the data converges with one another. As depicted by box 240, a common cell classifier is applied as follows. A dataset of all known events of the assay being used (referred to as “ATLAS) is applied to each common event cluster. In particular, each common event cluster is compared to all “ATLAS” data points and classified as one of our determined cell types. These events can then be enumerated. As depicted by box 250, the rare events undergo a filtering process, where each rare event candidate is compared to each common event cluster. This cleans the rare event candidate list for slide wide rarity, instead of regional rarity. As depicted by box 260, a rare cell classifier is applied as follows. The “ATLAS” dataset of all known events of the assay is applied. Each rare event candidate will be compared to all “ATLAS” data points and classified as one of our determined cell types. This step further filters out events that are “common.” The classified events can then be enumerated as certain cell types. Any event that is not classified within the “ATLAS” undergoes clustering and the aggregate information is collected and sent to the final report. As depicted by box 270, DAPI− event clustering proceeds as follows. All DAPI− events from the slide are collected, undergo dimensional reduction, and the hierarchical clustered into multiple groups. Each DAPI− group has the mean of the features of the events within the cluster. Each DAPI− event data is preserved as well as their position on the slide. The aggregated cluster information is sent to the report. As depicted by box 280, each common event cluster is represented in the report as 10 montages of sample even within the cluster as well as the count of all events within that cluster and their aggregate information. Each non-classified rare event cluster is represented similarly with 10 sample montages, the count of a events within the cluster, and their respective aggregate information. If the user wants to retrieve the individual event data or the events within a certain cluster, the user will send a command to the server to individually montage each event within the respective cluster. Similar to the non-classified rare events, the DAPI− event clusters are represented with 10 sample montages, the count of events within the cluster, and their respective aggregate information. If the user wants to retrieve the individual event data for the events within a certain cluster, the user will send a command to the server to individually montage each event within the respective cluster. The classified rare events, as well as any event within a cluster that the user sent to the server for individual event data collation, are individually montaged, easily sortable and Queryable, and are shown in a user interface that can give the user a holistic view of all rare events within the slide.

In some aspects, a method for evaluating a subject for cancer stage is provided is provided. This method may include having a liquid biopsy sample from the patient comprising biological structures; preparing a sample comprising a single layer of biological structures (“single biological structure layer sample”) from the liquid biopsy sample; staining the biological structures of the single biological structure layer sample with a fluorescence assay (e.g., immunofluorescence assay such as the Baseline assay set forth herein or any fluorescent assay); applying (e.g., determining) the biological structure identification system (s) set forth above; identifying the rare biological structures through their fluorescence and morphology; forming a biological structure identification bucket (“identification bucket”) based on the identified biological structure type, wherein each biological structure identification bucket contains the biological structure(s) that are similar in type; forming a set of identification buckets (“identification bucket set”) based on the identification buckets; comparing information related to the identification bucket set to that of the atlas; determining the disease afflicting the patient; and treating the patient. A processing system performs the following steps:
generate images of the one or more biological structures for the subject from the image data, detect and determine a plurality of features from the images or the image data, and form biological structure identification buckets from the plurality of features, each biological structure identification bucket identifying biological structures that are similar in type;
generate a subject profile of biological structure identification buckets for rare biological structures for the subject;
compare the subject profile with a set of predetermined cancer stage profiles of subjects having the cancer at a plurality of cancer stages; and
identifying a cancer stage for the subject by determining a predetermined cancer stage profile from the set of predetermined cancer stage profiles to which the subject profile is most similar. FIG. 1-5 illustrates the exemplary methods of this disclosure.

FIG. 1-5 provides a flow chart exemplifying a method for evaluating a patient with the methods provided herein. A human subject is presented for evaluation (Box 300). A liquid biopsy sample is obtained from the patient (Box 310). As depicted by Box 320, a single layer biological structure sample is prepared. A baseline assay for a cancer is used to stain the single layer biological structure sample (Box 330). The sample is loaded into the biological structure identification system (Box 340). As depicted in Box 350, fluorescence and morphology of biological structures are detected and determined. Images of the one or more biological structures for the subject are generated from the image data. A plurality of features are detected and determined from the images or the image data. Biological structure identification buckets are formed from the plurality of features with each biological structure identification bucket identifying biological structures that are similar in type (Box 360). A subject profile of biological structure identification buckets is generated for rare imaging events for the subject (Box 370). The subject profile is compared with a set of predetermined cancer stage profiles of subjects having the cancer at a plurality of cancer stage (Box 380). A cancer stage for the subject is identified by determining a predetermined cancer stage profile from the set of predetermined cancer stage profiles to which the subject profile is most similar (Box 390). The human patient can be treated accordingly with respect to cancer stage.

In the disease setting, in addition to these common immune cells, the liquid biopsy sample may further comprise rare cells that may actively escape or passively leak into the circulation and travel through the circulation, and may represent the disease.

Rare cells are defined as cells that are statistically distinct by their image analysis features. These rare cells are extracted by the following criteria: (a) after performing a bucketing analysis, the cells within the smallest population buckets are classified as rare; and (b) the cells within the cluster that is statistically deviant from the median value of all features from all cells are also classified as rare. The population of the rare cells may be lower than 5% of the total number of cells identified in the liquid biopsy sample. The population of the rare cells may be lower than 1% of the total number of cells identified in the liquid biopsy sample. The population of the rare cells may be lower than 0.1% of the total number of cells identified in the liquid biopsy sample.

The travel of the rare cells through the circulation may be with short half-lives or long half-lives. The rare cell travel may also include stopovers in various tissues along the way.

Representing the disease may mean that these rare cells may be (a) cancer cells as may be evidenced by their cancer genomic profiles and/or cancer protein markers; (b) tumor microenvironment cells that leak into circulation, wherein these cells may comprise epithelial cells, endothelial cells, mesenchymal cells, other stromal cells, cells that are in various transitional states, or a mixture thereof; (c) immune cells that may be responding to the tumor itself or cancer treatment; or (d) a mixture thereof.

The appearances of categories and classification of rare cells may be different across different cancers and stages of each cancer. Systems, methods and assays of this disclosure may identify various cellular subtypes both reproducibly for clinical practice while also enabling discovery of the unknown with an ability to detect a vast majority that have been implicated simultaneously in a unified experiment.

The subclasses of cells may be separated by protein and nuclear patterns as well as by cell morphology. The subclasses may be validated by downstream genomic or proteomic analyses, which might or might not be necessary for future clinical applications.

In another variation, one example relates to an approach to distinguish a substantially larger number of cellular groups using five markers. These markers are fluorescently protein antibodies or molecules labeled to four distinct fluorophores or fluorescent antibody. The computational method combines morphological differences as revealed by distinct fluorescence signatures to distinguish between at least twelve different rare cell subtypes, which may be present in the liquid biopsy sample. These rare cells are listed below.

This approach leverages both a new sample processing protocol reducing the five markers into four fluorescence channels and a novel computational method for classifying the different rare cell types via analysis of fluorescent microscopy images. Important for the success of this approach is the choice of marker combinations within and across fluorescent channels.

The computational approach is distinct from what everyone else is doing by putting ‘every event’ into a bucket of similar biological structures. Others look for specifics, for the known, which is a fundamental limitation of standard image analysis and of machine learning approaches as these would always ever only find the known. If on the other hand we force the computational method to accommodate every event on the slide defined by the existence of an imaging signal (in our current case it is fluorescent but it could also be brightfield), we can now cluster all events. As a next step, we allow for both common event clusters and rare event clusters. We in fact do not necessarily argue that all ‘cancer events’ are in rare clusters but instead we are effectively reducing the dimensionality of the total slide of millions of events with in itself hundreds of potential parameters, to a clustered framework that accommodates common (high frequency) and rare (low frequency) events. We know from the traditional CTC world that CTCs and by extension other disease associated events are typically rare.

Additional details of the invention are found in attached Exhibits A and B; the entire disclosure of which are hereby incorporated by reference.

The following examples illustrate the various embodiments of the present invention. Those skilled in the art will recognize many variations that are within the spirit of the present invention and scope of the claims.

### I. Multianalyte Liquid Biopsy to Aid the Diagnostic Workup of Breast

### Introduction

Accurate prognosis at the time of a diagnosis with early-stage breast cancer is a critical aspect of the diagnostic workup. Analytes in the blood-based liquid biopsy carry the opportunity for better characterization of the systemic burden of the disease during this clinical process. Breast cancer (BC) is the most common cancer in women globally and with 7.8 million cases diagnosed in the past 5 years, it is the world's most prevalent cancer overall (1-3). Approximately 94% of patients are initially diagnosed with early-stage BC, without evidence of macroscopic metastasis, however, despite the initial lack of detectable metastases and administration of subsequent treatments, 40% of the early-stage BC patients will go on to develop recurrence over their lifetime (4-9). Relapse, progression, and onset of distant metastasis (late-stage BC) have a significant negative impact on clinical outcomes, dropping the 5-year survival rate from 91% to less than 30% (1,3). Considering the impact on survival rates, it is vital that robust stratification of early-stage BC be made possible at the time of the initial diagnostic workup and throughout the course of the disease.

Currently, the standard screening method for BC is mammography, with a tissue biopsy to confirm diagnosis (3,4). In patients with biopsy confirmed cases of BC, tumor burden and treatment response are typically assessed by clinical evaluation of symptoms alongside imaging (4). While cross sectional advanced imaging is sometimes used to identify disease spread, it is expensive, often inconclusive, and fails to provide insight into the status and changes of the molecular profile of the tumor. Solid tissue biopsies have great utility in clinical care and can provide information on tumor biomarker and histological subtyping, molecular profiles, and advise treatment planning. Nevertheless, they have several caveats. First, primary tumors or metastatic lesions are not always easily accessible. Second, although solid biopsies provide valuable insights into the molecular signatures of the tumor, they are limited to the precise sampling area and could fail to capture the tumor heterogeneity (10-14). Third, and most crucial, solid biopsies are inherently incompatible with characterization of the subclinical systemic spread of the disease in addition to being challenging for longitudinal monitoring since they are painful, invasive, and always carry a potential risk to the patient (15-19).

Liquid biopsy (LBx), with a focus on peripheral blood, is a minimally-invasive method that can provide key information about the tumor and the systemic burden of the disease in the circulatory system (20,21). The utility of LBx for BC detection in the metastatic setting has been well-established with numerous clinical trials focusing on their utility to inform clinical decision-making and improve patient outcomes (22-28). Most of the LBx studies on BC focus on the presence of circulating tumor cells (CTCs), however, in the case of early-stage BC where CTC positive patients are scarce (29-33), more comprehensive analysis of tumor-related analytes in the LBx could be beneficial to assess the disease status. The third generation high-definition single cell assay (HDSCA3.0) workflow provides the opportunity to identify and characterize epithelial, mesenchymal, endothelial, and hematopoietic cells, as well as large extracellular vesicles (LEVs), building a platform capable of providing a more comprehensive overview of the circulating rare events and capturing the heterogeneity of the LBx (34).

In this study, we demonstrate the feasibility of using the HDSCA3.0 to stratify late-stage BC, early-stage BC, and normal blood donor status, using peripheral blood samples. We observe a distinctly higher presence of CTCs in the late-stage BC, compared to the early-stage and normal groups. Additionally, we determine that tumor-associated LEVs are found more frequently and in greater abundance in the early-stage BC group compared to late-stage and normal blood donor groups. In combination, this allows for both the stratification of cancer vs. normal and early- vs. late-stage BC with statistical confidence. Our results open the opportunity for a complementary LBx at the time of diagnostic workup for cancer detection, stage stratification, and disease monitoring.

### Materials and Methods

### Study Design

A total of 100 BC patients and 30 normal donors are included in this study. Cancer patients were recruited to the prospective Physical Sciences in Oncology study (PSOC-0068) entitled OPTImization of blood COLLection (OPTICOLL) (35). Here, we present a subset consisting of 74 patients clinically classified as early-stage and 26 patients clinically classified as late-stage BC at time of enrollment (Table 1). All cancer patients were enrolled between April 2013 and Jan. 17, 2017, at multiple clinical sites in the United States: Billings Clinic (Billings, MT), Duke University Cancer Institute (Durham, NC), City of Hope Comprehensive Cancer Center (Duarte, CA), and University of Southern California Norris Comprehensive Cancer Center (Los Angeles, CA). Patient recruitment took place according to an institutional review board approved protocol at each site and all study participants provided written informed consent (35,36).

The study schedules were coordinated and unified across the clinical sites. For patients included in this study with non-metastatic treatment naïve disease (early-stage BC), the blood draws were acquired prior to any treatment. Patients with metastatic disease (late-stage BC) had multiple blood specimens collected at the beginning of a new line of therapy, either as a first line of therapy or post-progression while on therapy for treatment of metastatic malignancy. A total of 10 normal blood donor samples were procured from the Scripps Clinic Normal Blood Donor Service and defined as individuals with no known pathology. Additionally, 20 age and gender matched normal donor samples were provided from Epic Sciences and defined as women between 45-82 yrs (median=57) with no known pathology. Normal donors will refer to the accumulation of both Scripps Clinic and Epic Sciences samples.

### Blood Collection and Processing

Approximately 8 mL peripheral blood was collected in 10-mL blood collection tubes (Cell-free DNA BCT, Streck) at the respective clinical site. Blood specimens were shipped to and processed at the Convergent Science Institute in Cancer (CSI-Cancer) at the University of Southern California within 24-48 hours of collection, as previously described (20). Upon receipt, all samples underwent red blood cell lysis and the remaining nucleated cell population was plated in a monolayer on custom-made cell adhesive glass slides (Marienfeld, Lauda, Germany), at approximately 3 million cells per slide. The prepped slides were subsequently incubated in 7% BSA, dried and stored at −80° C. (20, 35, 36).

### Immunofluorescence Assay

Two slides from each patient, corresponding to approximately 6 million nucleated cells, were thawed and subsequently stained using IntelliPATH FLX™ autostainer (Biocare Medical LLC, Irvine, CA, USA) in batches of 50 slides (46 patient slides [2 slides per patient] and 4 control slides) as previously described (20, 34, 36). All steps were performed at room temperature. Cells were fixed with 2% neutral buffered formalin solution (VWR, San Dimas, CA) for 20 min, nonspecific binding sites were blocked with 10% goat serum (Millipore, Billerica, MA) for 20 min. Slides were subsequently incubated with 2.5 ug/mL of mouse anti-human CD31 monoclonal antibody (Ab) (clone: WM59, MCA1738A647, BioRad, Hercules, CA) preincubated with 100 ug/mL of goat anti-mouse IgG monoclonal Fab fragments (115-007-003, Jackson ImmunoResearch, West Grove, PA) for 4 hr. After incubation with CD31-Fabs, cells were permeabilized using 100% cold methanol for 5 min. Cells were then incubated with an Ab cocktail consisting of mouse anti-human pan-cytokeratin (PanCK) mAbs (clones: C-11, PCK-26, CY-90, KS-1A3, M20, A53-B/A2, C2562, Sigma, St. Louis, MO), mouse anti-human CK19 mAb (clone: RCK108, GA61561-2, Dako, Carpinteria, CA), mouse anti-human CD45 Alexa Fluor®647 mAb (clone: F10-89-4, MCA87A647, AbD serotec, Raleigh, NC), and rabbit anti-human vimentin (VIM) mAb (clone: D21H3, 9854BC, Cell Signalling, Danvers, MA) for 2 hr. Slides were then incubated with Alexa Fluor®555 goat anti-mouse IgG1 antibody (A21127, Invitrogen, Carlsbad, CA) and counterstained with 4′,6-diamidino-2-phenylindole (D1306, ThermoFisher, Waltham, MA) for 40 min. Slides were then mounted with an aqueous mounting media to preserve cellular integrity for further downstream analysis.

### Image Acquisition and Feature Extraction

After staining, the slides were imaged using automated high-throughput fluorescence scanning microscopy at 100× magnification, resulting in 2304 image frames per slide, as previously reported (20). Exposure times and gain for PanCK, VIM, CD45/CD31, and DAPI (DNA) channels were determined computationally by the scanner control software to normalize the background intensity levels across all slides. Using customized EBImage (4.12.2) software and the R scripting language for image analysis, cells were segmented, and their cellular and nuclear descriptors were extracted as previously described (34).

### Rare Event Identification, Classification, and Analysis

Rare events were detected by the third-generation of our computational algorithm for unsupervised clustering, as previously described (34). In brief, this approach allows for the classification of cells into common and rare groups based on principal component analysis of cells' morphometric features and subsequent hierarchical clustering (FIG. 2-1). Additionally, the algorithm identified large DAPI-|PanCK+ events (1-10 μm in diameter) to be classified as LEV candidates, as previously demonstrated (37).

Rare cells were then further classified into 8 classes based on the combinations of immunofluorescent marker expression in 3 categories: PanCK, VIM, CD45/CD31. Four categories showed no expression of cytokeratins but were determined positive for either VIM or CD45/CD31, or determined positive or negative for both. Enumerations of the cellular categories were done by trained analysts who determined the final enumeration per cell type.

Finally, the frequency of rare events (CTCs and LEVs) for each category was reported as concentration of rare cells per ml (mean, median, range), calculated by measuring the total number of nucleated cells per two slides, estimated using DAPI-stained nuclei count, against the total complete blood count of the received sample.

### Morphometric Comparison

The computational approach uses EBImage to segment cells and extract quantitative cellular and nuclear features (34). For our morphometric analysis, we utilized the extracted features to further analyze the identified rare cells. Features correspond to cell size and eccentricity, nucleus size and eccentricity, immunofluorescent intensity of the DAPI, PanCK, VIM, CD45/CD31 channels, and the ratios of all combinations of these features to one another. Values for the immunofluorescent channels are reported as the mean signal over cell area, normalized per slide to interval 0-1.

### Statistical Analysis

Statistical two-sided analyses were performed using R (Version 4.1.1., Boston, MA). Groups were compared using Kruskal-Wallis (one-way ANOVA on ranks) for non-parametric rank-based dependence between multiple groups to compare whether the distributions have a median shift greater than the null hypothesis, and student's t-test to determine if there is a significant difference between the means of two groups, for all analyses. P values below 0.05 were considered statistically significant. No correction was conducted as the comparisons were planned comparisons. Pearson correlation was used to evaluate the relationship between study groups.

### Machine Learning Model

The primary goal of this study was to determine the ability of HDSCA3.0 rare cell detection to stratify normal donor, early-stage BC, and late-stage BC into distinct groups based on the rare cellular events detected using the LBx approach. While this stratification was initially performed using statistical analysis on the cell counts, we explored the ability of using machine learning models with the target variable of disease state. We used the manual enumeration recorded as event counts per ml per fluorescent channel type. To overcome discrepancies in the sample size, we randomly oversampled the late-stage BC group to match the size of the early-stage BC cohort. Similarly, we oversampled the normal group to match the size of the combined BC groups. To ensure we were not biasing the dataset by oversampling two groups, we also performed combinations of random undersampling of early-stage and oversampling normal, as well as undersampling both early- and late-stage groups.

For the model, we tested random forest, logistic regression, and naïve bayes algorithms using Python 3 (Python Software Foundation, https://www.python.org/) and Orange 3.0 data-mining toolbox in Python (38). Model comparison was done by measuring the accuracy, sensitivity, specificity, and AUC (area under the ROC curve) to evaluate performance. In all comparisons, random forest was the top performing algorithm.

To determine the stratification efficiency of the LBx using HDSCA3.0, a random forest algorithm was used to develop models to predict disease state classification. We built a random forest model with 10 trees. Our random forest model was trained, validated, and tested using data from 296 samples (74 early-stage, 74 late-stage, and 148 normal donors). Training and validation of the model was performed on ˜75% of the dataset through random selection (111 BC and 111 normal donors for cancer vs. normal/56 early-stage BC and 55 late-stage BC for early vs. late), using 10-fold cross validation. Testing of the model was performed on the remaining ˜25% of the dataset (37 BC and 37 normal donors for cancer vs. normal/18 early-stage BC and 19 late-stage BC for early vs. late), thereby maintaining the class distribution across training/validation/test sets.

### Results

### Patient Demographics and Clinical Baseline

A total of 155 blood draws from 130 participants, with 74 (56.9%) treatment-naive, nonmetastatic early-stage patients, 26 (20%) metastatic late-stage, and 30 (23.1%) normal donors, were included in this study. All participants were female. Patients' demographics are provided in Supplemental Table 1. The total sample set included 310 slides each containing approximately 3 million nucleated cells that were processed and analyzed for rare event detection (Methods).

### Identification, Enumeration, and Morphometric Analysis of Rare Cells

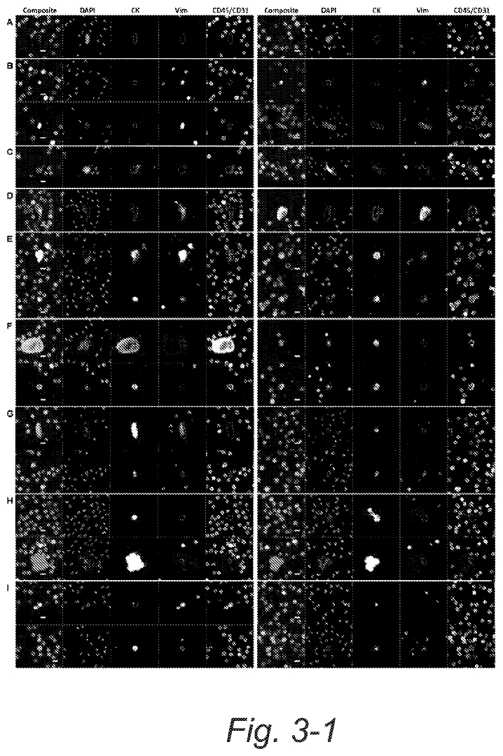

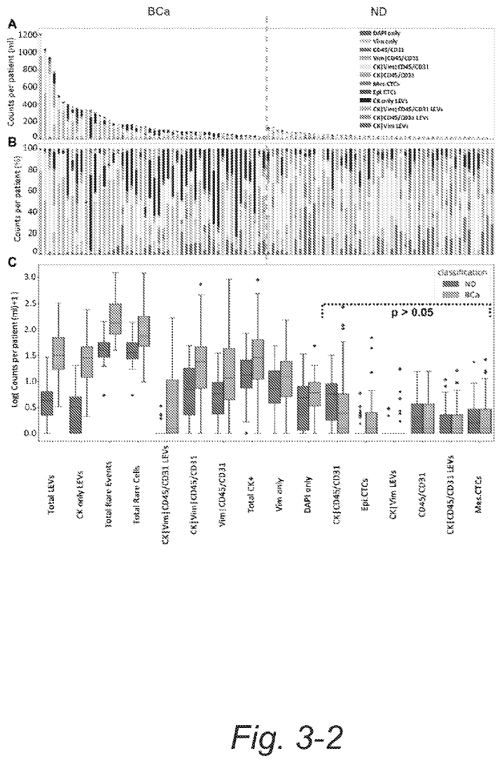

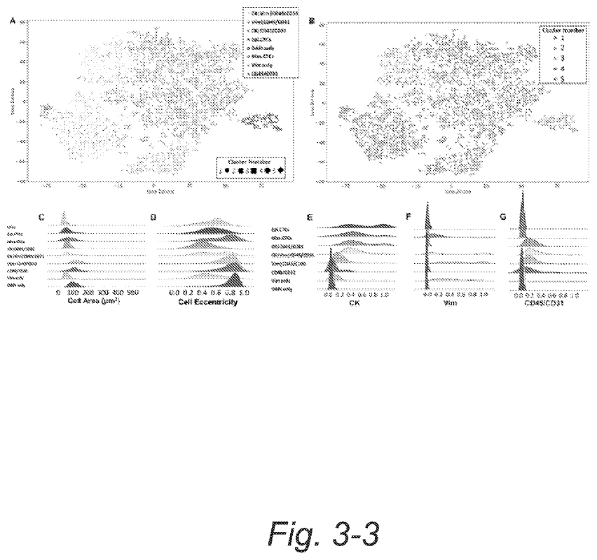

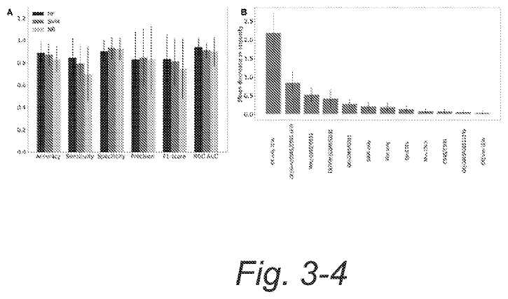

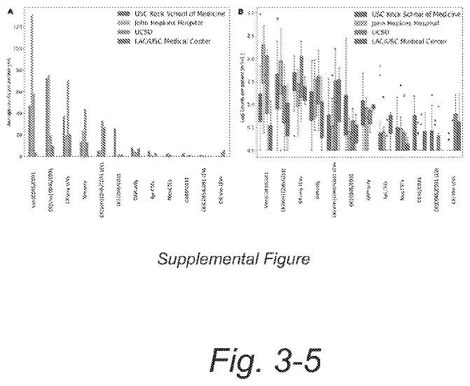

We identified and categorized candidate rare cells using an automated rare cell detection workflow followed by manual enumeration based on the four-channel immunofluorescence staining corresponding to DAPI, PanCK, VIM, CD45/CD31, and cellular morphology (FIG. 2-2). Enumeration of total rare cells revealed a significantly higher overall count in late-stage BC patients (mean=48.67, median=36.36, range=8.01-383.32 cells/ml) compared to early-stage BC (mean=36.19, median=23.06, range=1.58-284.54 cells/ml; p=0.01), and late-stage BC compared to normal donors (mean=14.27, median=12.89, range=0-37.43 cells/ml; p=0.0015×10⁻⁰³). A significant difference was also observed between the early-stage BC patients and normal donors (p=0.0012) (FIG. 2-3.A-B).

CTCs that were identified as DAPI+|PanCK+ were defined as epi.CTCs and enumerated for normal donor, early-stage BC, and late-stage BC samples. The epi.CTC enumeration of all samples revealed a median of 0 cells/ml (mean=2.66, range=0-50.10 cells/ml). For the late-stage group, 75% of patients had at least one epi.CTC (mean=6.75, median=2.02, range=0-50.10 cells/ml), compared to only 27% of early-stage patients (mean=0.77, median=0, range=0-12.13 cells/ml; p=0.0011×10⁻⁰⁴). Late-stage patients had a significantly higher level of epi.CTCs than the normal donor group (mean=0.39, median=0, range=0-2 cells/ml; p=0.0038×10⁻⁰³). No significant difference in the epi.CTCs was observed between the early-stage BC and the normal donor groups. (FIG. 2-3.A-B).

VIM+ CTCs (mes.CTCs) were identified as DAPI+|PanCK+|VIM+. For all samples we observed a median of 0 cells/ml (mean=1.27, range=0-16.42). The late-stage BC group revealed a significantly higher overall count of mes.CTCs (mean=2.52, median=1.02, range=0-16.42 cells/ml), in comparison with the early-stage BC (mean=0.91, median=0, range=0-7.06 cells/ml; p=0.0019) and the normal donor (mean=0.55, median=0, range=0-5 cells/ml; p=0.0024) groups. No significant difference was observed between the normal donor and early-stage BC groups) (FIG. 2-3.A-B).

Additional candidate CTCs include PanCK+|CD45/CD31+ (double positive CTC) and PanCK+|VIM+|

CD45/CD31+ (triple positive CTC) cells. No significant difference was observed between the levels of double positive CTCs between the groups. The triple positive CTCs were found at significantly higher frequencies in both the early-stage BC (mean=12.80, median=1.80, range=0-240.04 cells/ml; p=0.008) and the late-stage BC (mean=4.34, median=2.07, range=0-40.56 cells/ml; p=0.014) compared to the normal donor (mean=1.56, median=0, range=0-17.062 cells/ml) group. No significant difference was observed in the comparison between the early- and late-stage groups (FIG. 2-3.A-B).

Other detectable rare cells include morphologically distinct VIM+|CD45/CD31+|DAPI+, CD45/CD31+|DAPI+, DAPI+, and VIM+|DAPI+ cells. The VIM+|DAPI+ only cells showed a significant increase in the late-stage group (mean=14.43, median=4.74, range=0-266.82 cells/ml), compared to the early-stage (mean=3.84, median=1.44, range=0-27.81 cells/ml; p=0.00056) and the normal donor (mean=1.72, median=0.93, range=0-12.10 cells/ml; p=0.0031×10⁻⁰²) groups (FIG. 2-3.A-B).

Morphological analysis was conducted on the identified rare cells based on extracted image features from EBImage. A visual representation of the identified rare cells based on their morphometric features has been provided as a uniform manifold approximation and projection (UMAP) figure (FIG. 2-3.C), as well as a low-dimensional TSNE plot (Supplemental FIG. 2-8). In the UMAP projection, the majority of manually classified cells cluster together by channel type classification, indicating robust manual classification across the cohort. The CTCs detected in late-stage BC samples demonstrated higher PanCK expression, measured by normalized signal intensity, (mean=0.80, median=0.87, range=0 to 0.60) than their early-stage BC counterparts (mean=0.61, median=0.56, range=0.44 to 0.74, p=0.00015) (FIG. 2-3.D).

A correlation analysis between the frequency of classified rare cell categories was conducted for all samples and no strong correlation was found (FIG. 2-3.E).

### Identification and Enumeration of Tumor-Associated LEVs

LEVs, classified as DAPI-|PanCK+ events were most prevalent in the early-stage BC group, with 94% of patients having at least one LEV per ml, compared to 60% in the late-stage group (FIG. 2-4.A). The frequency of LEVs was overall elevated in the early-stage BC group (mean=43.78, median=20.31, range=0 to 400.52), compared to the late-stage BC (mean=2.92, median=1.37, range=0 to 21.91, p=0.0027×10⁻⁰¹²) and the normal donor (mean=0.99, median=0, range=0 to 6.73, p=0.0024×10⁻¹⁰) groups. A significant difference was also observed between the late-stage BC and the normal donor groups (p=0.018) (FIG. 2-4.B). Identified LEVs fell into the size range (5.89-14.02 micrometer in diameter), representing the smallest rare event category (FIG. 2-4.C). The marker expression profile of classified LEVs were similar to that of epi.CTCs, with some expression of VIM and CD45/CD31 detected, as shown in FIG. 2-4.D. Scaled plots depicted in FIG. 2-4.E indicate a higher overall presence of LEVs in the early-stage group, compared to the late-stage and normal donor. A correlation analysis between the frequency of classified rare cell categories and LEVs was conducted for all samples and no strong correlation was found.

Correlations with Clinical Outcome

In the patient population with identified hormone receptor (HR) and end-of-therapy status (44 early-stage/57% and 12 late-stage/46%) (Supplemental Table 1, FIG. 2-7), we evaluated whether the identified rare events are associated with clinical markers and patient outcomes. In the early-stage BC group, the overall median time from diagnosis to follow-up was 27 months (range=8 to 99, n=44), with no reported mortalities.

Our results indicate a significantly higher frequency of LEVs in the early-stage BC group with the last follow-up status of “alive, free of disease” (mean=46.10, median=20.25, range=0 to 400.52 LEVs/ml, n=39) in comparison to those with “alive, active cancer” (mean=18.03, median=11.89, range=7.41 to 32.88 LEVs/ml; p=0.047, n=5) (FIG. 2-5.A). Levels were also found to be elevated in patients with human epidermal growth factor receptor 2 (HER2) negative (mean=48.22, median=21.46, range=0 to 400.52 LEVs/ml, n=37) compared to HER2 positive (mean=15.13, median=11.34, range=7.41 to 46.41 LEVs/ml; p=0.026, n=7) tumor status (FIG. 2-5.B). No significant correlation was observed between HER2 tumor status and follow-up patient status in the early-stage BC patients.

In the late-stage BC group, the overall median time from diagnosis to follow-up was 19.5 months (range=1 to 41, n=14), with no cases reported to be cancer-free. We found significantly higher epi.CTC levels in group with the follow-up status of “deceased, active cancer on day of death” (mean=21.96, median=17.68, range=0 to 50.10 cell/ml, n=6), compared with “alive, active cancer” (mean=1.37, median=1.48, range=0 to 3.40 cell/ml; p=0.045, n=8).

Epi.CTC counts were also found to be elevated in BC patients with estrogen receptor (ER) positive (mean=14.78, median=2.18, range=0 to 50.10, n=9) compared to ER negative (mean=1.93, median=2.44, range=0 to 3.83, p=0.072, n=5) tumor status. The same relationship was also detected between the progesterone receptor (PR) positive (mean=20.33, median=13.70, range=0 to 50.10, n=6) and PR negative (mean=2.59, median=1.69, range=0 to 10.15, p=0.086, n=8) patients, although both levels did not reach statistical significance. No significant relationship was observed between ER/PR tumor status and follow-up patient status in the late-stage BC patients. No significant difference was observed between HER2 tumor status and epi.CTC levels.

### Patient Level Classification Model

The random forest model exhibited acceptable performance, as measured by the ROC/confusion matrix, between normal vs. cancer and early-stage vs. late-stage comparisons (FIG. 2-6.A-B). LEV enumeration was the strongest predictor for correctly classifying into late, early, and normal, followed by epi.CTC enumeration. (FIG. 2-6.C). Our normal vs. cancer model reached 0.99 AUC in classification and an F1 score (0.98%), exhibiting robust performance. Additionally, our early-stage vs. late-stage model reached 0.91 AUC, with similar performance for F1 score (0.86%) (FIG. 2-6.D).

### Discussion

In this study, we set out to stratify late-stage BC, early-stage BC, and normal donor peripheral blood samples based on rare circulating events identified using the HDSCA3.0 LBx platform. We utilized 5 biomarkers to identify and distinguish rare circulating events as epithelial, mesenchymal, endothelial, or hematological origin. Using this comprehensive profiling without prior enrichment, we were able to observe events in all samples, allowing for robust stratification with both manual classification and mathematical model building approaches. We were able to detect reproducible patterns in the enumeration of rare cells and LEVs. These reproducible patterns separate the relevant groups of cancer vs. normal control and early-stage cancer vs. late-stage cancer with high accuracy. Our findings demonstrate the feasibility to provide robust and reproducible detection of rare circulating events in peripheral blood draws and to stratify late-stage BC, early-stage BC, and normal donor samples.

Since metastasis is the most common cause of cancer mortality (1), earlier detection and precise diagnosis of existent and early tumor dissemination is imperative to improving patient outcomes. In our study, we found a statistically significant increase of CTCs in patients of the late-stage compared to early-stage BC groups. Previous studies have attributed the higher frequency of CTCs in late-stage BC patients to the dissemination of tumor (39), therefore the lower incidence rate observed in the early-stage cancer setting could be explained by the organ-confined nature of the disease and lack of widespread metastasis. Previous work has demonstrated a link between CTC burden in late-stage BC and progression free survival (40), however, administration of treatment has been shown to affect the abundance of CTCs (41). In this study of late-stage BC patients, with draws taken either on and off therapy, we were able to detect epi.CTCs in 75% of the samples and observe negative association of epi.CTC count with overall survival. Therefore, our results using a high-sensitivity non-enrichment technology demonstrate that epi.CTCs may still be detected, and provide prognostic value prior to the initiation of therapy, as well as during treatment.

Despite advances in the LBx field, the low abundance of CTCs, especially in early-stage cancer, remains a challenge for establishing precise diagnosis and prognosis in this setting. Furthermore, tumors are complex and are comprised of heterogeneous cell types, with CTCs that are defined by dual positivity for EpCAM and Cytokeratin only representing a fraction of the total tumor cells responsible for dissemination and relapse (42). Motivated by these prior observations, this next generation LBx was designed to identify and characterize the tumor heterogeneity in the circulatory system. By including eight rare cell categories, we were able to observe the heterogeneous phenotypes in circulation and to use these multiple LBx analytes to stratify the samples according to disease status with high statistical significance.

Detection of LEVs represent a promising new LBx analyte (37). Our results demonstrate a statistically higher overall presence of tumor-associated LEVs in the early-stage BC group, compared to the late-stage BC group and the normal donors. The high level of LEVs in the early-stage BC patients could be explained by the presence of the primary tumor, since these early-stage BC patient samples were collected prior to any treatment, at which time the patient still had their primary tumor intact. This contrasts with the late-stage patients, who are more likely to have had their primary tumor removed prior to the time of blood draw. Tumor associated LEVs have been described as a component of the tumor microenvironment (45), and primary tumors have been shown to harbor more cellular heterogeneity in comparison to metastatic lesions which are mostly composed of tumor cells (46). Additionally, previous findings have implicated extracellular vesicles for their role in facilitating pre-metastatic niche preparation (47,48). Tumor progression and metastasis requires the acquisition of invasive traits within the primary tumor alongside the generation of a permissive microenvironment at distant metastatic sites. Previous studies have found that in the case of BC, extracellular vesicles can initiate organ-specific pre-metastatic niche preparation (49). These results suggest that there is an additional possibility that LEVs are secreted into circulation in pre-metastatic early-stage disease from the primary tumor to facilitate the preparation of metastatic niches and are less inclined to be present in late-stage disease where the metastatic sites are well-established. Our study demonstrates that detection of LEVs, when applied alongside rare cell enumeration, provides a more sensitive and specific LBx analysis.

The OPTICOLL study was originally designed to provide a comprehensive analysis of pre-analytical variables of LBx (35,36) and is providing a platform for discovery using sample preparation methods that have been previously validated. A limitation of this study is the number of patients with sufficient follow-up that we were able to include. The results of this study should however provide sufficient feasibility to conduct larger trials and higher patient recruitment as the next step towards clinical utility. Both the use of additional lineage markers and the inclusion of LEVs in addition to CTCs has significantly advanced our ability to separate the patient groups. The patients with sufficient follow up did not yet include plasma preparation for cell-free analysis, which one would expect to also add value. However, despite the current limitations, we were able to observe a highly significant difference in the LBx analytes between breast cancer patients and normal controls, and between the late-stage and early-stage BC samples collected. While the current observations are consistent with prior hypotheses of various liquid biopsy analytes, we expect these results will trigger further model system experiments to continue exploration of the early and late-stage implications of LEVs in particular as well as the design of additional trials to define the clinical utility as a potential adjunct to the diagnostic workup.

A more comprehensive profiling of the LBx as demonstrated here has the potential to complement the current diagnostic workup following a positive screening test. The current NCCN guidelines do not recommend systemic imaging such as FDG-PET scanning for the majority of early-stage patients as most patients will receive some form of adjuvant treatment (53). However, LBx findings, such as the frequencies of LEVs and CTCs, may provide diagnostic and prognostic information that would impact the utility of adjuvant systemic therapy in subsets of patients. LBx may also identify those patients who have occult secondary tumors as evidenced by persistence of LEVs following primary surgery or predict whether post-operative patients are more or less likely to benefit from adjuvant radiotherapy. For patients at risk of breast cancer, LBx may also have a role as an adjunct to radiologic screening for breast cancer by stratifying the Breast Imaging-Reporting and Data System (BI-RADS) category 3 patients into categories 2 or 4 based on LBx results. Such a combined approach may reduce the patient anxiety associated with indeterminate mammography results and reduce the need for 6 months call back imaging. Each of these hypotheses require testing in large scale prospective trials.

II. Characterization of Cellular and Acellular Analytes from Pre-Cystectomy Liquid Biopsies in Patients Newly Diagnosed with Primary Bladder Cancer

### 1. Introduction

Bladder cancer (BCa) is the tenth most common cancer in the world, representing 3% of all new cancer cases [54]. Urothelial carcinoma (˜90%) is the most frequent BCa histology diagnosed in the U.S., and can be subdivided by stage, grade, and subtype (conventional or variant morphology) [55]. Less common types include squamous (2-5%), adenocarcinoma (2%), and neuroendocrine (1%), as well as other rare tumors (<1%). Tumors that are confined to the lamina propria of the bladder are termed non-muscle invasive BCa (NMIBC; Ta, Tis (carcinoma in situ), T1), while those that invade the muscularis propria are called muscle invasive BCa (MIBC, T2-T4), an advanced stage with life threatening consequences requiring surgical management. BCa is highly lethal once cells have spread from the primary tumor to surrounding tissues and distant organs [56]. Cystectomy, the surgical removal of the bladder, is used to treat most BCa patients, as it offers the best chance of cure. The procedure can be performed alone or in combination with other treatments and can be considered a first-line intervention in cases of superficial tumors with severe anaplasia.

We have previously reported on the clinically observed patterns of relapse following cystectomy. Metastases developed in 29% of patients (n=812), resulting in a 5-year overall survival rate of 20.4%, compared to 78.6% in those without relapse (n=1,983) [57,58]. Most metastatic progression occurs within the first 24 months. In another study, information theory and machine learning algorithms were employed to create predictive models around this BCa database, in which the primary predictors of recurrence and survival after radical cystectomy were determined to be pathologic T stage and subgrouping into localized or metastatic conditions [56]. Clinical T stage had a lower predictive signal than the true pathologic T stage. This loss of valuable information may especially affect those cases in which there is an underestimation of disease severity prior to surgery [59]. This recognizes the limitation of current clinical staging at the time of diagnosis and highlights the importance of precision cell and tissue analysis in differentiating patients by outcome prior to and following surgical intervention.

The early relapse in primary BCa patients undergoing cystectomy may be attributed to the presence of pre-existing subclinical metastatic disease in these patients [57]. Current prominent methods for detection, diagnosis, and surveillance of the disease are based on urine cytology and cystoscopy. Urine cytology, while non-invasive, approximately yields a low sensitivity of 38% and a specificity of 98% [60]. On the other hand, cystoscopy has a higher sensitivity between 65-90% depending on the subtype but is a highly invasive procedure with significant inter- and intra-observer variation in tumor stage and grade [61]. Thus, there is great need for improving the current clinical paradigm of diagnostic workup and treatment planning. We hypothesized that the liquid biopsy as a biomarker of systemic disease may be diagnostic of subclinical metastatic disease and prognostic of early relapse. If proven correct, it could serve as a surrogate marker to guide the addition or use of alternative therapy as opposed to surgical intervention alone in patients diagnosed with BCa. A comprehensive analysis of the blood-based liquid biopsy may assist in solving complex clinical problems by tracking cellular evolution and phenotypic populations, revealing treatments that are not efficacious for specific patients, thus developing a stratification system in order to avoid unnecessary surgical intervention.

Circulating tumor cells (CTCs) shed by the tumor are often detectable in the peripheral blood (PB) of cancer patients and have been associated with poor prognosis and early relapse [61-64]. Busetto et al. observed a strong correlation between the detection of CTCs by CellSearch® and the time to first recurrence [62]. Furthermore, in a meta-analysis of 2161 BCa patients from 30 published articles, Zhang et al. showed that the number of CTCs detected in the PB correlated with tumor stage, histological grade, metastasis, and regional lymph node metastasis [63]. These studies indicate that the presence of CTCs in the PB is an independent predictive indicator of poor outcomes for BCa patients. The work presented here is based on a third-generation comprehensive liquid biopsy [65]. This non-enrichment based, high-content direct imaging methodology is capable of providing both visualization and characterization of a broad range of CTCs that are present in circulation, along with molecular parameters (DNA and protein) at both the cellular and acellular (large extracellular vesicles [LEVs] and cell-free DNA [cfDNA]) levels. We have previously reported the value of single-cell genomic analysis conducted on this platform showing compatibility with clinical practice [65-67].

The third generation high-definition single cell assay (HDSCA3.0) liquid biopsy workflow [68-70] was designed for rare cell identification with immunocytochemistry [18] along with downstream molecular characterization in order to deliver diagnostic pathology-quality data for clinical decision making [66,67,72-74]. The primary objective of the present study was to investigate the prognostic significance of CTCs in BCa patients from PB samples taken prior to cystectomy. Secondary objective was to assess the association between CTC presence and known clinical data metrics such as clinical or pathological staging and histological subtype. This study aims at establishing evidence for the clinical utility of the liquid biopsy in BCa with the future goal of predicting metastatic relapse post-cystectomy and enable clinical intervention that can lead to improved outcomes.

### 2. Materials and Methods

### 2.1 Study Design

This was a multiple institution prospective study of patients diagnosed with BCa in which PB samples were collected before cystectomy and prior to any procedures. Eligible patients underwent cystectomy for surgical removal of the primary tumor from the bladder. University of Southern California's Keck School of Medicine (Keck; n=25) samples were collected between January and November 2020. Samples from the University of California San Diego (UCSD; n=9), Johns Hopkins Hospital (JHH; n=13), and LAC/USC Medical Center (LAC; n=3) were collected between January 2016 and November 2017. The Keck patient subset has prospectively collected clinical, radiologic, and pathologic data elements as well as a limited amount of follow-up data. For this cohort, recurrence is defined as any clinical recurrence majority shown radiologically, either symptomatic or not. Patient recruitment took place according to an institutional review board approved protocol at each site, and all study participants provided written informed consent. Here we present the liquid biopsy analysis from a total of 50 BCa patients. Additionally, 50 normal donor (ND) samples from individuals with no known pathology were provided from Epic Sciences (San Diego, CA).

### 2.2. Blood Sample Processing

PB samples were collected in 10 ml blood collection tubes (Cell-free DNA, Streck) and processed by the Convergent Science Institute in Cancer (CSI-Cancer) at the University of Southern California within 24-48 hours as previously described [71]. Briefly, samples underwent red blood cell lysis, followed by plating the entire nucleated cell fraction on custom glass slides (Marienfeld, Lauda, Germany) at approximately 3 million cells per slide prior to long-term cryostorage at −80° C. and rare cell analysis.

### 2.3. Blood Sample Staining and Imaging

For HDSCA analysis, each test consisted of two slides generated from the PB sample for an average of 0.74 ml blood analyzed. Slides were processed at room temperature using the IntelliPATH FLX™ autostainer (Biocare Medical LLC, Irvine, CA, USA) as previously described [65]. Briefly, samples were stained with 2.5 ug/ml of a mouse IgG1 anti-human CD31:Alexa Fluor®647 mAb (clone: WM59, MCA1738A647, BioRad, Hercules, CA) and 100 μg/ml of a goat anti-mouse IgG monoclonal Fab fragments (115-007-003, Jackson ImmunoResearch, West Grove, PA), permeabilized using 100% cold methanol, followed by an antibody cocktail consisting of mouse IgG1/Ig2a anti-human cytokeratins (CKs) 1, 4, 5, 6, 8, 10, 13, 18, and 19 (clones: C-11, PCK-26, CY-90, KS-1A3, M20, A53-B/A2, C2562, Sigma, St. Louis, MO), mouse IgG1 anti-human CK 19 (clone: RCK108, GA61561-2, Dako, Carpinteria, CA), mouse anti-human CD45:Alexa Fluor®647 (clone: F10-89-4, MCA87A647, AbD Serotec, Raleigh, NC), and rabbit IgG anti-human vimentin (Vim) (clone: D21H3, 9854BC, Cell Signaling, Danvers, MA). Lastly, slides were incubated with Alexa Fluor® 555 goat anti-mouse IgG1 antibody (A21127, Invitrogen, Carlsbad, CA) and 4′,6-diamidino-2-phenylindole (DAPI; D1306, ThermoFisher) prior to mounted with a glycerol-based aqueous mounting media. Samples were imaged using automated high-throughput fluorescence scanning microscopy at 10× objective magnification generating 2,304 frames images per fluorescence channel per slide.

### 2.4. Rare Event Detection and Classification

As previously reported [65], rare cell candidates were detected using a custom computational methodology termed OCULAR (Outlier Clustering Unsupervised Learning Automated Report). Fluorescent images were used to segment each cell using the “EBImage” R package (EBImage_4.12.2) and extract 761 quantitative morphometric parameters based on the nuclear and cytoplasmic morphology and biomarker expression (CK, Vim, CD45/CD31) in a 4-channel immunofluorescence assay (DAPI, AlexaFluor® 488, AlexaFluor® 555, AlexaFluor® 647). Additionally, the algorithm identified DAPI-negative CK-positive events into a separate report to be classified as large extracellular vesicle (LEV) candidates [73].

Manual reporting was conducted on the identified events to check for signal intensity and distribution, as well as morphology. Images of candidate rare events were presented to a hematopathologist-trained technical analyst for analysis and interpretation. Rare events were classified into 12 categories (8 cellular, 4 LEV) based on the combination of immunofluorescent marker expression in the previously reported 4 channels. Epithelial-like CTCs (epi.CTCs) were classified as cells that were CK-positive, Vim-negative, and CD45/CD31-negative, with distinct appearing nucleus by DAPI morphology as previously described [65,71]. Epi.CTCs expressing Vim were classified as mesenchymal-like CTCs (mes.CTCs). White blood cell (WBC) counts of whole blood were determined automatically (Medonic M-series Hematology Analyzer, Clinical Diagnostic Solutions Inc., Fort Lauderdale, FL) and the number of WBCs detected by the assay per slide was used to calculate the actual amount of blood analyzed per test so that results are presented as fractional values of events/ml.

LEV candidates were positive for CK with variable Vim and CD45/CD31 expression. LEVs were identified through the OCULAR methodology outlined above with careful identification for those that were either free-floating or in close proximity to cells. Due to the close proximity of the cell-attached LEVs, OCULAR interpreted both as a single cellular event. Manual classification to separate these two entities as individual rare events was employed to correct for the computational oversimplification of OCULAR. Further, corrections included excluding any halos, bubbles, or light refractions resembling the morphology of LEVs (round and membranous) when examining frames of patient samples through the CK channel. A maximum threshold of three LEVs per frame was used to rule out CK-positive junk particles that may have landed on the slide during processing

### 2.5. Statistical Analysis

Statistical significance was determined at a p-value ≤0.05. To perform statistical analysis of the clinical, radiologic, and pathologic data, we used two statistical tests: Spearman's rank correlation coefficient [75] and the Mann-Whitney U test, also known as the Wilcoxon rank sum test [76,77]. The Spearman rank test was used to calculate the correlation between continuous variables as we are not strictly evaluating the degree of linear relationship, but rather the degree of monotonic relationship between the two target variables. In addition, it was also non-exclusively applied to evaluate the correlation between continuous variables and categorical variables that have a well-defined ordinal encoding and multiple outcomes. For example, the clinical T stage encoded such that the available classifications (T0, Tis, Ta, T1, T2a, T3b, T4a) were assigned to ordinal values from 0 to 6. To evaluate the correlation between continuous and categorical data without a well-defined ordinal encoding, we also performed the Wilcoxon rank sum test.

The Wilcoxon rank sum test determines whether two samples are likely to derive from the same population, is appropriate for small datasets, and does not require that the data be paired or normally distributed [78]. This nonparametric test is calculated based on the ranks (or order) of the numerical variables, making it robust with respect to outliers. For categorical variables that can have more than two classifications, the Wilcoxon rank sum test is calculated between all possible classification pairs. For example, the correlation between total rare cell count vs clinical predominant cancer cell type (Urothelial, Other, No Tumor) is calculated for all combinations: Urothelial vs Other, Urothelial vs No Tumor and Other vs No Tumor. All statistical tests were performed in Python (version 3.8.5) with the Scipy library (version 1.5.0).

To visualize the morphometrics of detected cellular events, a two-dimensional tSNE (t-distributed stochastic neighbor embedding) was used [79]. To aid the identification of clusters in the tSNE, a clustering algorithm was used. Specifically, we applied agglomerative clustering imported from the sklearn library version 0.23.2 [80]. For the clustering parameters, we used Ward linkage and an Euclidian distance metric [81].

### 2.6. Patient Level Classification Modeling

Classification models were used to test whether BCa patients can be discerned from NDs utilizing liquid biopsy data alone (i.e., whether one has distinct rare event populations when compared to the other). The python library sklearn version 0.23.2 was used to develop the machine learning models [80]. Two slides each from 50 ND samples were collected to mirror the 50 BCa patient samples. For each individual, the data utilized in the classification models was the counts for each cell and event classification per ml of blood averaged across both slides. Three different classification models (random forest [RF], support vector machine [SVM] and naive Bayes [NB]) were tested to produce a binary outcome indicating whether an individual is within the BCa or ND category. We employed a 5-fold cross validation method to test each model architecture in which the dataset was divided into five equal folds of 20 individuals. Each fold is then used as a test set for a model built with the remaining four, yielding five models for each or RF, SVM, and NB (i.e., 15 total models). We employed a grid search algorithm to find optimal hyperparameters for the RF and SVM. Final model metrics are averages across all models of the same type.

### 3. Results

A total of 50 patients with primary BCa were accrued for this study, each providing a single PB sample obtained prior to cystectomy. Site specific liquid biopsy data is provided in supplemental FIG. 3-5. Three patients within the Keck subset withdrew consent after surgery and are not included in the statistical analyses between liquid biopsy and clinical data. Clinical and demographic data metrics were collected for the Keck subset (n=22) and are provided in Table 1. At the time of data collection, 2 of the patients had recurred and 1 was deceased. ND information was limited to participant age (median 57, range 45-82, mean 58.9).

TABLE 1
Clinical demographics for Keck subset of patients.

| | | |
|---|---|---|
| Age | 71.4 | (53.4-86.1) |
| BMI | 24.9 | (21.2-36.9) |
| Hgb | 11.1 | (5.1-15.0) |
| HCT | 34.2 | (18.3-46.3) |
| WBC | 7.6 | (4.8-20.4) |
| Platelets | 201.5 | (57-387) |
| BUN | 22.5 | (13-70) |
| Creatinine | 1.2 | (0.5-3.1) |

| Race | Caucasian | 20 |
|---|---|---|
| | Asian | 2 |
| Gender | Male | 18 |
| | Female | 4 |
| Smoker | Previous | 14 |
| | Current | 4 |
| | Never | 4 |
| Neoadjuvant Chemo | Yes | 10 |
| | No | 12 |
| Surgical Procedure | Anterior Exenteration | 1 |
| | Radical Cystectomy | 4 |
| | Robotic Radical | 17 |
| | Cystectimy | |
| Urinary Diversion | Studer | 9 |
| | Ileal Conduit | 11 |
| | Indiana Pouch | 2 |
| Pure Urothelial (CS/PS) | | 7/4 |
| Predominant Histology | No Tumor | 2/9 |
| (CS/PS) | Urothelial | 17/11 |
| | Other | 3/1 |
| | Plasmacytoid | 0/1 |
| Squamous (CS/PS) | Absent | 16/12 |
| | Present | 2/1 |
| | NA | 4/9 |
| Glandular (CS/PS) | Absent | 16/12 |
| | Present | 2/1 |
| | NA | 4/9 |
| Neuro (CS/PS) | Absent | 18/12 |
| | Present | 1/1 |
| | NA | 3/9 |
| Subgroup (CS/PS) | OC | 16/15 |
| | EV | 4/3 |
| | N+ | 2/4 |
| T Stage (CS/PS) | T0 | 2/9 |
| | Ta | 2/0 |
| | Tis | 1/4 |
| | T1 | 1/2 |
| | T2a | 11/0 |
| | T2b | 0/1 |
| | T3a | 0/3 |
| | T3b | 2/1 |
| | T4a | 3/2 |
| N Stage (CS/PS) | NX | 2/0 |
| | N0 | 19/18 |
| | N2 | 1/4 |

Abbreviations: CS, clinical staging; PS, pathological staging; OC, organ confined; EV, extravesical; N+, node positive; BMI, body mass index; Hgb, hemoglobin; HCT, hematocrit; WBC, white blood cell; BUN, blood urea nitrofgen.

### 3.1. Liquid Biopsy Analysis Prior to Cystectomy

A complete blood cell count was taken at CSI-Cancer prior to blood processing. For the 50 BCa samples included here, there was a median WBC count of 6.75 (range 3.3-25; mean 7.5) million cells/ml PB. For all BCa samples, total rare event (total cells and LEVs) detection had a median of 132.67 events/ml (range 38.11-1,220.51; mean 230.33). For ND samples, total rare event detection had a median of 38.50 events/ml (range 4.39-141.55; mean 47.86). A significant difference was observed between the BCa patients and ND (p-value <0.0001).

### 3.2. Rare Cell Characterization

We have identified 8 cellular categories defined by nuclear DAPI signal and rely on the expression of the different biomarkers in each channel. A gallery of CTCs and graphical representation of the frequency of each rare event identified per test for each patient sample is shown in FIGS. 3-1 and 3-2. Total rare cell detection for the BCa samples had a median of 74.61 cells/ml (range 8.75-1,213.69; mean 178.40). The ND samples presented with a median rare cell detection of 34.46 cells/ml (range 4.39-137.03; mean 43.21). A statistically significant difference in total rare cell detection was observed between the BCa patients and ND samples (p-value <0.0001).

Total CK-positive cells were detected with a median of 27.59 cells/ml (range 0-895.72; mean 79.36) from all BCa samples. The ND samples had a median of 12.90 cells/ml (range 0-83.24; mean 18.96). There was a statistically significant difference in total CK-positive cell detection between BCa patient and ND samples (p-value=0.0093). Only 1 BCa patient (2%) did not present with CK-positive cells at the time of sample collection. Using a threshold of positivity of >5 cells/ml, a total of 44 samples (88%) were positive for CK expressing cells. The frequency of CK-positive cells detected within the total rare cell population varied. Overall, there was a median frequency of 30.2% (range 0-97%; mean 36%) in the BCa samples.

Epi.CTCs were detected with a median of 0 cells/ml (range 0-27; mean 1.2) from BCa patient samples. Mes.CTCs were detected with a median of 0 cells/ml (range 0-25.12; mean 2.33) from BCa patient samples. There was no statistically significant difference in epi.CTCs/ml or mes.CTCs/ml observed between BCa patient and ND samples.

Additional candidate CTCs detected include CK|CD45/CD31 (median 1.44 cells/ml; range 0-267.84; mean 13.76) and CK|Vim|CD45/CD31 (median 23.19 cells/ml; range 0-729.44; mean 60.09). Other detectable rare cells include morphologically distinct Vim|CD45/CD31 (median 10.51 cells/ml; range 0-919.24; mean 68.36), CD45/CD31 only (median 0 cells/ml; range 0-14.49; mean 1.89), DAPI only (median 5.00 cells/ml; range 0-46.86; mean 6.76), and Vim only (median 11.18 cells/ml; range 0-149.57; mean 22.04). There was a statistically significant difference between BCa patient and ND samples in cellular enumeration of Vim|CD45/CD31 (p-value=0.0018), CK|Vim|CD45/CD31 (p-value=0.0003), Vim only (p-value=0.0406), DAPI only (p-value=0.0430). The biological significance of these cellular populations has not been determined.

The most prevalent cell types observed in the PB of BCa patients prior to cystectomy were Vim|CD45/CD31 (median 15.19%; range 0-80.53%; mean 28.64%) and CK|Vim|CD45/CD31 cells (median 22.99%; range 0-79.49%; mean 26.10%), followed by Vim only cells (median 14.02%; range 0-81.13%; mean 21.94%). Out of all the rare cells detected across patient samples, Vim|CD45/CD31 cells constituted 45.24%, CK|Vim|CD45/CD31 cells constituted 31.05% and Vim only cells constituted 10.74%. We identified a positive correlation between mes.CTC and CK|Vim|CD45/CD31 (spearman coefficient=0.58, p-value <0.001), as well as two other cellular categories (Vim only [spearman coefficient=0.358, p-value=0.01], CK|CD45/CD31 [spearman coefficient=0.292, p-value=0.040]). This suggests that the cellular populations are associated with each other and represent the heterogeneity of the disease.

To visualize the cellular subgroups and their similarities with respect to morphometrics we used 8 key measures. The first four are obtained from the median immunofluorescence intensity of DAPI, CK and CD45/CD31 channels. The second set of four are the area and eccentricity for the cell and the nucleus. The morphometrics were visualized by a two-dimensional tSNE plot shown in FIG. 3-3. Each rare cell is represented with a single point, which is color coded based on its classification. Furthermore, to aid the interpretation of the cellular clusters, agglomerative clustering was applied to separate the cells in five clusters based on the same set of morphometrics. The plot markers were adjusted accordingly to match each cell to the corresponding cluster, as determined by the algorithm.

The channel-type classified cellular populations had observable morphological heterogeneity which is displayed in FIG. 3-3. Morphological analysis indicates multiple distinct cellular populations independent from biomarker expression. The DAPI only and Vim only cells cluster distinctly from the other channel-type groups by their morphology forming cluster number 3 and 5 respectively. The epi.CTC, mes.CTC, and CK|CD45/CD31 cells cluster together in cluster number 4, suggesting these are morphologically related. The CK|Vim|CD45/CD31 cell population has multiple distinct morphological subtypes, with a subset of cells that cluster with the epi.CTCs, mes.CTCs, and CK|CD45/CD31 cells. Another CK|Vim|CD45/CD31 subset is morphologically similar to the Vim|CD45/CD31 cells, which were strongly positively correlated (spearman coefficient=0.40, p-value=0.004). This suggests high heterogeneity of the CK|Vim|CD45/CD31 population, which may represent multiple distinct cellular phenotypes related to BCa.

### 3.3. LEV Detection

LEVs were classified by DAPI negativity, CK signal positivity and distribution, as well as morphology. Total LEV detection for the BCa patient samples had a median of 30.91 LEVs/ml (range 2.22-319.08; mean 51.92). The ND samples presented with a median of 3.34 LEVs/ml (range 0-27.91; mean 4.65), which was significantly lower than that detected in the BCa samples (p-value <0.0001). In BCa patient samples, LEVs were detected either alone (n=740; 44.6%) or in close proximity to cells (n=918; 55.4%). In ND samples, these LEV populations totaled 85 (45.9%) and 100 (54.1%), respectively.

CK only LEVs were detected in all BCa patients with a median of 27.06 LEVs/ml (range 1.08-235.92; mean 37.80). CK|Vim|CD45/CD31 LEVs were also detected in 27 patients (54%) with a cohort median of 1.05 LEVs/ml (range 0-163.95; mean 11.60). A positive correlation was observed between CK|Vim LEVs and CK|Vim|CD45/CD31 LEVs (spearman coefficient=0.47, p-value=0.001). Both of these LEV populations were detected at a significantly higher level in BCa patient samples than ND samples (p-value <0.0001 for both). The observed LEVs represent additional tumor heterogeneity and a new potential analyte to monitor disease status.

The detection of LEVs was not associated with the detection of epi.CTCs or mes.CTCs. We observed a negative correlation between Vim|CD45/CD31 cells and CK only LEVs (spearman coefficient=−0.39, p-value=0.005). Additionally, a negative correlation was found between CK|Vim LEVs and DAPI only rare cells (spearman coefficient=−0.28, p-value=0.05).

3.4. Keck Cohort with Clinical Data

Correlation analysis was used to determine the relationship between the various liquid biopsy analytes and the clinical/demographics metrics collected for the Keck subset of patients (n=22). Here, we report only the significant correlations, whereas a complete table of all comparisons can be found in the supplemental. A negative correlation was detected between BMI and the Vim only cells/ml (spearman coefficient=−0.41, p-value=0.05), as well as age and the DAPI only cells/ml (spearman coefficient=−0.59, p-value <0.001). WBC count correlated with CK|CD45/CD31 cells/ml (spearman coefficient=0.47, p-value=0.02) and CK|Vim|CD45/CD31 cells/ml (spearman coefficient=0.46, p-value=0.03). Platelet count at the time of sample collection correlated with total rare events/ml (spearman coefficient=0.57, p-value <0.001), total CK expressing cells/ml (spearman coefficient=0.47, p-value=0.02), mes.CTCs/ml (spearman coefficient=0.48, p-value=0.02), total LEVs/ml (spearman coefficient 0.61, p-value <0.001), CK only LEVs/ml (spearman coefficient=0.63, p-value <0.001). Creatinine blood measurements correlated with CK only LEVs/ml (spearman coefficient=0.43, p-value=0.04).

Clinical T stage was negatively correlated with CK|CD45/CD31 LEVs/ml (spearman coefficient=−0.62, p-value <0.001). Pathological T stage was negatively correlated with total rare events/ml (spearman coefficient=−0.50, p-value=0.01) and total rare cells/ml (spearman coefficient=−0.53, p-value=0.01). Those patients with Tis had significantly more rare cells/ml than those patients with T3a pathological staging (Wilcoxon=−2.12, p-value=0.03). The significance of the other channel-type rare cells have yet to be determined. Additionally, patients with Tis had a significantly greater CK only LEVs/ml than patients with T3a pathological staging (Wilcoxon=−2.12, p-value=0.03). This suggests that LEVs could be an analyte for early disease.

Total cells/ml, total LEVs/ml, and CK+LEVs/ml negatively correlated with recurrence (spearman coefficients=−0.44, −0.42, −0.42, respectively; p-value <0.05). The potential for recurrence is low as this prospective study had a median follow-up time since surgery of 9 months (range: 6-17) and additional time is warranted for progression/survival data to mature.

### 3.5. Patient Level Classification Modeling

Statistical tests and predictive modeling were used to discern the BCa population from NDs. According to the Wilcoxon rank sum test, the counts/ml detected in NDs belong to different populations than the corresponding samples of BCa for multiple rare event classifications and groups. According to the classification models, the BCa patients and NDs contained distinct cell populations that allowed for stratification, as evidenced by their overall accuracies. The RF, SVM, and NB architectures had average accuracies across their five respective models of 89%+/−9.7%, 87%+/−9.8%, and 83%+/−11.2%, respectively. This corresponds to incorrectly predicting 11 (BCa=5, ND=6), 13 (BCa=8, ND=5), and 17 (BCa=12, ND=5) individuals across each of the models. When looking at the receiver operating characteristic (ROC) curves, the RF yielded an average AUC of 0.94+/−0.09, as compared to 0.91+/−0.07 for SVM and 0.90+/−0.13 for NB. Among the three architectures tested, the RF achieved the highest sensitivity of all models (84%+/−18%), but the lowest specificity (90%+/−9%). Comparatively, the SVM and NB had sensitivities of 79%+/−17% and 70%+/−25% and specificities of 93%+/−10% and 92%+/−10%, respectively.

For the RF, the top three most important events for discerning BCa from ND were CK only LEVs, CK|Vim|CD45/CD31 LEVs, and Vim|CD45/CD31 cells (See FIG. 3-4). In fact, six of the top seven events are all statistically different across the two groups, which intuitively makes sense. It is important to note, however, that the most important event, CK only LEVs, is approximately 2.6 times and 4.2 times as important as the following two, respectively. This clearly indicates and supports our previous findings on the distinct differences between BCa and ND liquid biopsies.

### 4. Discussion

We have detected liquid biopsy analytes unique to patients diagnosed with BCa prior to cystectomy. More precise clinical diagnostic tools are warranted in the context of BCa to predict response to therapy and monitor minimum residual disease to minimize metastatic progression. This study documents several important findings for liquid biopsy analysis for patients with BCa undergoing cystectomy: (i) CTCs and LEVs are detected in the PB, (ii) there is a high heterogeneity of CTCs, and (iii) liquid biopsy analytes correlate with clinical data elements. The liquid biopsy is a useful non-invasive tool for the discovery of cancer related biomarkers to represent the complex process of tumorigenesis. Our findings suggest that CTC and LEV analysis from the liquid biopsy should be further investigated as an inclusion in BCa patient management.

In summary, our study found that rare cells can be detected in BCa PB samples (median 74.61 cells/ml) as well as ND samples (median 34.46 cells/ml). When specifically considering CK-positive cells, BCa samples presented a median of 27.59 cells/ml while ND samples presented a median of 12.90 cells/ml. This study also found that LEVs can be detected in BCa samples, at a significantly higher count than in ND samples (median 30.91 vs 3.34 LEVs/ml). Across all BCa samples, both epi.CTCs and mes.CTCs were observed in only 34% and 40% patients, respectively. However, other candidate CTCs were detected at higher frequencies which include CK|CD45/CD31 (median 1.44 cells/ml) and CK|Vim|CD45/CD31 (median 23.19 cells/ml). Additionally, our study found that multiple liquid biopsy analytes both positively and negatively correlated with clinical data metrics, including clinical and pathological T stage, as well as recurrence. For example, patients with Tis disease had significantly more rare cells and CK only LEVs than those with T3a disease.

There are several methods to detect bladder cancer, some more technically challenging and maintaining invasive requirements for the procedure, but different methods have varying degrees of accuracy which depends on the method's sensitivity and specificity. By having a foundational understanding of the interpretation of sensitivity and specificity, healthcare providers will understand outputs from current and new diagnostic assessments, aiding in decision-making and ultimately improving healthcare for patients. Cystoscopy is invasive and uncomfortable for patients due to the technical requirements of the procedure; but is still the most accurate diagnosis method for BCa (sensitivity 68-100%, specificity 57-97%; [82]. Urine cytology is a non-invasive liquid biopsy approach, and when high-grade tumors are considered, the sensitivity is high (84%), but the sensitivity decreases to 16% in NMIBC, precluding its use in the detection of low-grade lesions [83]. Here we show that in a mixed cohort (NMIBC and MIBC), applying classification models using liquid biopsy data, we achieved an average sensitivity of 78% and specificity of 92% for the identification of BCa patient samples. We set out to use the liquid biopsy for detection of subclinical metastasis prior to surgical resection. While this remains our primary goal, the data also supports the general consideration of the liquid biopsy for screening and diagnostic work-up of BCa.

The liquid biopsy might be an indicator of early disease dissemination with micrometastases, and assessment prior to cystectomy is therefore crucial. CellSearch® CTCs were detectable in 8/44 NMIBC patients at diagnosis (18%) in which the presence of CTCs was associated with a shorter time to first recurrence [84]. Using the HDSCA3.0 workflow we detected epi.CTCs in 38% and mes.CTCs in 46% of BCa patients presented here, however there was no statistical difference between the same type of cells detected in the ND samples. Detection of CTCs prior to cystectomy in BCa patients has been shown to serve as evidence of progressing disease which may predict the appearance of a macroscopic lesion in a longer-term period, therefore the patients with low CTC counts before cystectomy are hypothesized to have a low risk of recurrence and are thus good candidates for cystectomy [64]. Additional time is needed to monitor the progress of the BCa patients in this study and determine if our hypothesis is correct.

A heterogeneous population of rare cells was observed in the PB of BCa patients prior to cystectomy. Here we identified 8 categories of rare cells based on the expression of 4 biomarkers (CK, Vim, CD45/CD31), but further cellular stratification could be conducted using morphometric parameters as these categories include a mixture of cell types as seen by morphological analysis. Since the total rare cell count/ml was correlated with pathological T staging (spearman coefficient=−0.53, p-value=0.01), we conclude that the rare cells detected are indeed related to disease status. This is evidence for the circulation of multiple CTC populations and other rare cells, possibly from the tumor microenvironment (TME), as measures of tumor burden and disease state. Furthermore, since the HDSCA3.0 workflow detects rare cells beyond the epi.CTCs, the negative association between total rare cells/ml and tumor stage may be driven by the high frequency of cells other than CTCs that may represent the tumor microenvironment (TME). We hypothesize that rare cell populations expressing Vim|CD45/CD31 includes circulating endothelial cells (CECs). In a prior publication, we showed that CECs (CD138|von Willebrand Factor positive, CD45 negative) in PB samples were morphologically distinct from the surrounding WBCs, and CEC count was significantly higher in myocardial infarction patients than that of the healthy control [85]. The presence of CECs in the PB may be a novel way to assess vascular function in BCa patients, potentially as markers of altered vascular integrity or even direct contributors to tumor formation (i.e., angiogenesis). Further characterization is warranted to understand the biological significance of each channel-type cellular population, but this study highlights the promise of the liquid biopsy for early risk stratification of BCa patients, prediction of treatment response, and early detection of metastatic relapse.

Here we show that circulating LEVs have been detected in an enrichment-free liquid biopsy approach, representing a promising new analyte for BCa care. Tumor heterogeneity is further seen in the 4 different LEV categories detected. The results presented here demonstrated a statistically higher overall presence of tumor-associated LEVs in BCa patients prior to cystectomy compared to the NDs (median 30.91 LEVs/mL vs. 3.34 LEVs/mL, respectively), most likely due to the presence of the primary tumor. Exosomes contain a number of analytes (nucleic acids, proteins, and metabolites) which strongly reflect the parental cell properties, making them a promising alternative to CTCs or circulating tumor DNA (ctDNA) as biomarkers of disease. In a study of extracellular vesicles (EV; size 30-200 nm) detected from urine, BCa patients had higher concentration of EVs in the urine when compared with healthy controls, with a sensitivity of 81.3% and a specificity of 90.0% in the discrimination of BCa patients against healthy controls [86]. This supports the utility of LEVs in the diagnostic work-up for BCa clinical care. In prostate cancer, LEVs detected in the PB using the same workflow were 1.9 times as frequent as CTCs and shared a similar protein signature [73]. Here we show that LEVs are associated with BCa tumorigenesis and may be useful diagnostic and prognostic biomarkers. Further characterization of the LEVs detected here will validate their neoplastic origin and association with the BCa disease state.

Molecular characterization of the rare events detected in this study will elucidate their potential role in BCa tumorigenesis. Molecular profiling through genomic and proteomic analysis of a patient's liquid biopsy will have value in enabling the discovery of novel drivers of growth and metastasis that help direct individual treatment or identify potential new treatment targets. Using the HDSCA workflow, we have the unique opportunity for a comprehensive analysis of the liquid biopsy [66,67,74,87-90]. Previous studies have used single-cell sequencing and targeted multiplexed proteomic analysis to characterize both circulating rare and common cells detected by the HDSCA workflow in a variety of clinical scenarios [66,67,74,87,88,91]. Additionally, cfDNA genomic analysis is possible for a more comprehensive view of the liquid biopsy. Multiple prior studies indicate that ctDNA is detectable in plasma of BCa patients, and high levels of ctDNA are associated with progression and metastatic disease [92-95]. Chalfin et al. show that CTC and ctDNA provide complementary information in urothelial carcinomas [96]. The ability to characterize tumor heterogeneity using a single platform with comprehensive single-cell DNA, single-cell multiplexed targeted proteomics, and cfDNA analysis could provide precision diagnostics from the time of initial diagnosis for patients with BCa. Future research aims to establish evidence towards the clinical utility of the liquid biopsy in BCa to predict metastatic relapse post cystectomy and enable clinical intervention to lead to improved outcomes.

### 5. Conclusions

This study establishes evidence for the clinical utility of the liquid biopsy in BCa with the future goal of predicting metastatic relapse post-cystectomy and enabling clinical intervention that can lead to improved outcomes. Here we show the identification of rare cells and LEV frequencies unique to BCa patients, with distinct populations within and across patients underscoring the heterogeneity of liquid biopsy profiles. Further, the high specificity and sensitivity metrics of the prediction models demonstrate the stratification of BCa patients from ND using this methodology. While further investigation is needed to elucidate the predictive power of these analytes with respect to recurrence, the findings from this study show the liquid biopsy as a promising clinical tool for early-stage BCa patients.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Siegel, R. L., Miller, K. D., Fuchs, H. E. & Jemal, A. Cancer statistics, 2021. CA Cancer J. Clin. 71, 7-33 (2021
2. American Cancer Society. Cancer Facts & Figures 2021. Atlanta: American Cancer Society; 2021.
3. American Cancer Society. Breast Cancer Facts & Figures 2019-2020. Atlanta: American Cancer Society, Inc. 2019.
4. AJCC (American Joint Committee on Cancer) Cancer Staging Manual; 8th edition, 3rd printing, Amin M B, Edge S B, Greene F L, et al (Eds), Springer, Chicago 2018.
5. Mariotto A B, Etzioni R, Hurlbert M, Penberthy L, Mayer M. Estimation of the Number of Women Living with Metastatic Breast Cancer in the United States. Cancer Epidemiol Biomarkers Prev. 2017; 26(6):809-815
6. Pan, H. et al. 20-year risks of breast-cancer recurrence after stopping endocrine therapy at 5 Years. N Engl J Med. 377(19), 1836-1846 (2017).
7. Colleoni M, Sun Z, Price K N, et al. Annual hazard rates of recurrence for breast cancer during 24 years of follow-up: results from the international breast cancer study group trials I to V. J Clin Oncol 2016; 34:927-35
8. Sestak I, Dowsett M, Zabaglo L, Lopez-Knowles E, Ferree S, Cowens J W, et al. Factors predicting late recurrence for estrogen receptor-positive breast cancer. J Natl Cancer Inst. 2013; 105: 1504-11. 2.
9. Nishimura R, Osako T, Nishiyama Y, Tashima R, Nakano M, Fujisue M, et al. Evaluation of factors related to late recurrence—later than 10 years after the initial treatment—in primary breast cancer. Oncology. 2013; 85:100-10.
10. Gerlinger M, Rowan A J, Horswell S, Math M, Larkin J, Endesfelder D, et al. Intratumor heterogeneity and branched evolution revealed by multiregion sequencing. N Engl J Med. 2012; 366(10):883-92. 4.
11. Hinohara K, Polyak K. Intratumoral heterogeneity: more than just mutations. Trends Cell Biol. 2019; 29(7):569-79.
12. Dagogo-Jack I, Shaw A T. Tumour heterogeneity and resistance to cancer therapies. Nat Rev Clin Oncol. 2018; 15(2):81-94. https://doi.org/10.1038/nrclinonc.2017.166.
13. Polyak, K. Heterogeneity in breast cancer. J. Clin. Invest. 121, 3786-3788 (2011)
14. Zardavas, D., Irrthum, A., Swanton, C. & Piccart, M. Clinical management of breast cancer heterogeneity. Nat. Rev. Clin. Oneal. 12, 381-394 (2015).
15. Alieva, M., van Rheenen, J., & Broekman, M. (2018). Potential impact of invasive surgical procedures on primary tumor growth and metastasis. Clinical & experimental metastasis, 35(4), 319-331. https://doi.org/10.1007 Isl 0585-018-9896-8
16. Griffiths, J. I., Chen, J., Cosgrove, P. A. et al. Serial single-cell genomics reveals convergent subclonal evolution of resistance as patients with early-stage breast cancer progress on endocrine plus CDK4/6 therapy. Nat Cancer 2, 658-671 (2021). https://doi.org/10.1038/s43018-021-00215-7
17. Harbeck, N. et al. Breast cancer. Nat. Rev. Dis. Prim. 5, 66 (2019).
18. Almendro, V., Marusyk, A. & Polyak, K. Cellular heterogeneity and molecular evolution in cancer. Annu. Rev. Pathol.-Mech. Dis. 8, 277-302 (2013).
19. Fazel, R. et al. Exposure to low-dose ionizing radiation from medical imaging procedures. N Engl. J. Med. 361, 849-857 (2009).
20. Marrinucci D. et al. Fluid biopsy in patients with metastatic prostate, pancreatic and breast cancers. Phys Biol. February 2012; 9(1): 016003. doi: 10.1088/1478-3975/9/1/016003 PMCID: PMC3387996
21. Kuhn P. & Bethel K. EDITORIAL: A fluid biopsy as investigating technology for the fluid phase of solid tumors Phys Biol. February 2012; 9(1): 010301. doi:10.1088/1478-3975/9/1/010301.
22. Bidard F C, Peeters D J, Fehm T, et al. Clinical validity of circulating tumour cells in patients with metastatic breast cancer: a pooled analysis of individual patient data. Lancet Oncol. 2014 April; 15(4):406-414.
23. Budd G T, Cristofanilli M, Ellis M J, et al. Circulating tumor cells versus imaging-predicting overall survival in metastatic breast cancer. Clin Cancer Res. 2006 Nov. 1; 12(21):6403-6409.
24. Giuliano M, Giordano A, Jackson S, et al. Circulating tumor cells as prognostic and predictive markers in metastatic breast cancer patients receiving first-line systemic treatment. Breast Cancer Res. 2011 Jun. 15; 13(3):R67.
25. Bidard F-C, Jacot W, Dureau S, et al. Abstract GS3-07: clinical utility of circulating tumor cell count as a tool to chose between first line hormone therapy and chemotherapy for ER+ HER2− metastatic breast cancer: results of the phase III STIC CTC trial. Cancer Res. 2019; 79(4Supplement):GS3-07-GS3-07.
26. Hayes D F, Cristofanilli M, Budd G T, et al. Circulating tumor cells at each follow-up time point during therapy of metastatic breast cancer patients predict progression-free and overall survival. Clin Cancer Res. 2006 Jul. 15; 12(14 Pt 1):4218-4224.
27. Liu M C, Shields P G, Warren R D, et al. Circulating tumor cells: a useful predictor of treatment efficacy in metastatic breast cancer. J Clin Oncol. 2009 Nov. 1; 27(31):5153-5159.
28. Smerage J B, Barlow W E, Hortobagyi G N, et al. Circulating tumor cells and response to chemotherapy in metastatic breast cancer: SWOG S0500. J Clin Oncol. 2014 Nov. 1:32(31):3483-3489.
29. Krishnamurthy S, Cristofanilli M. Singh B. et al. Detection of minimal residual disease in blood and bone marrow in early-stage breast cancer. Cancer 2010; 116:3330-7.
30. Tibbe A G, Miller M C, Terstappen L W. Statistical considerations for enumeration of circulating tumor cells. Cytometry A 2007; 71:154-62.
31. Rack B, Schindlbeck C, Jiickstock J, et al. Circulating tumor cells predict survival in early average-tohigh risk breast cancer patients. J Natl Cancer Inst 2014; 106:dju066.
32. Pierga J Y, Bidard F C, Mathiot C, et al. Circulating tumor cell detection predicts early metastatic relapse after neoadjuvant chemotherapy in large operable and locally advanced breast cancer in a phase II randomized trial. Clin Cancer Res 2008; 14:7004-10.
33. Lucci A, Hall C S, Lodhi A K, et al. Circulating tumour cells in non-metastatic breast cancer: a prospective study. The Lancet Oncology 2012; 13:688-95
34. Chai, S., Matsumoto, N., Storgard, R., Peng, C. C., Aparicio, A., Ormseth, B., Rappard, K., Cunningham, K., Kolatkar, A., Nevarez, R., Tu, K. H., Hsu, C. J., Malihi, P., Com, P., Zurita, A., Hicks, J., Kuhn, P., & Ruiz-Velasco, C. (2021). Platelet-Coated Circulating Tumor Cells Are a Predictive Biomarker in Patients with Metastatic Castrate-Resistant Prostate Cancer. Molecular Cancer Research: MCR, 10.1158/1541-7786.MCR-21-0383. https://doi.org/10.1158/1541-7786.MCR-21-0383
35. Rodriguez-Lee M, Kolatkar A, McCormick M, Dago, AE, Kendall J, Carlsson N A, Bethel K, Greenspan E, Hwang S, Waitman K, Nieva J, Hicks J, Kuhn P. Effect of blood collection tube type and time to processing on the enumeration and high-content characterization of circulating tumor cells using the high-definition single cell assay. Arch Pathol Lab Med 2018; 142: 198-207
36. Shishido S N, Welter L, Rodriguez-Lee M, Kolatkar A, Xu L, Ruiz C, Gerdtsson A S, Restrepo-Vassalli S, Carlsson A, Larsen J, Greenspan E J, Hwang S E, Waitman K R, Nieva J, Bethel K, Hicks J, Kuhn P. Pre-analytical variables for the genomic assessment of the cellular and acellular fractions of the liquid biopsy in a cohort of breast cancer patients. J Mol Diag, Volume 22, Issue 3, Pages 319-337, March 2020
37. Gerdtsson A S, Setayesh S M, Malihi P D, Ruiz C, Carlsson A, Nevarez R, Matsumoto N, Gerdtsson E, Zurita A, Logothetis C, Com P G, Aparicio A M, Hicks J, Kuhn P. Large Extracellular Vesicle Characterization and Association with Circulating Tumor Cells in Metastatic Castrate Resistant Prostate Cancer. Cancers (Basel). 2021 Mar. 2; 13(5):1056. doi: 10.3390/cancers13051056. PMID: 33801459; PMCID: PMC7958848.
38. Demsar J, Curk T, Erjavec A, Gorup C, Hocevar T, Milutinovic M, Mozina M, Polajnar M, Toplak M, Staric A, Stajdohar M, Umek L, Zagar L, Zbontar J, Zitnik M, Zupan B (2013) Orange: Data Mining Toolbox in Python, Journal of Machine Learning Research 14(August): 2349-2353.
39. Dasgupta A, Lim A R, Ghajar C M. Circulating and disseminated tumor cells: harbingers or initiators of metastasis?Mol Oncol. 2017 January; 11(1):40-61. doi: 10.1002/1878-0261.12022. PMID: 28085223; PMCID: PMC5423226.
40. Janni W J, Rack B, Terstappen LWMM, Pierga J-Y, Taran F-A, Fehm T, Hall C, de Groot M R, Bidard F-C, Friedl T W P (2016) Pooled Analysis of the Prognostic Relevance of Circulating Tumor Cells in Primary Breast Cancer. Clin Cancer Res 22, 2583-2593. 10.1158/1078-0432.ccr-15-1603
41. van Dalum, G., van der Stam, G. J., Tibbe, A. G., Franken, B., Mastboom, W. J., Vermes, I. Terstappen, L. W. (2015). Circulating tumor cells before and during follow-up after breast cancer surgery. International Journal of Oncology, 46, 407-413.https://doi.org/10.3892/ijo.2014.2694
42. Dasgupta, A., Lim, A. R., & Ghajar, C. M. (2017). Circulating and disseminated tumor cells: harbingers or initiators of metastasis?. Molecular oncology, 11(1), 40-61. https://doi.org/10.1002/1878-0261.12022 43. Pantel K, Speicher M R. The Biology of Circulating Tumor Cells. Oncogene (2016) 35(10):1216-24. doi: 10.1038/onc.2015.192
44. Wu, S., Du, Y., Beckford, J. et al. Upregulation of the EMT marker vimentin is associated with poor clinical outcome in acute myeloid leukemia. J Transl Med 16, 170 (2018). https://doi.org/10.1186/s12967-018-1539-y
45. Maacha S, Bhat A A, Jimenez L, Raza A, Haris M, Uddin S, Grivel J C. Extracellular vesicles-mediated intercellular communication: roles in the tumor microenvironment and anti-cancer drug resistance. Mol Cancer. 2019; 18(1):55.
46. Seyfried, T. N., & Huysentruyt, L. C. (2013). On the origin of cancer metastasis. Critical reviews in oncogenesis, 18(1-2), 43-73. https://doi.org/10.1615/critrevoncog.v18.il-2.40
47. Minciacchi V R, Freeman M R, Di Vizio D. Extracellular vesicles in cancer: exosomes, microvesicles and the emerging role of large oncosomes. Semin Cell Dev Biol. 2015; 40:41-51.
48. Meehan B, Rak J, Di Vizio D. Oncosomes—large and small: what are they, where they came from? J Extracell Vesicles. 2016; 5:33109.
49. Chin, A. R., & Wang, S. E. (2016). Cancer Tills the Premetastatic Field: Mechanistic Basis and Clinical Implications. Clinical cancer research: an official journal of the American Association for Cancer Research, 22(15), 3725-3733. doi: 10.1158/1078-0432.CCR-16-0028
50. Ruiz, C., Li, J., Luttgen, M. S., Kolatkar, A., Kendall, J. T., Flores, E., Topp, Z., Samlowski, W. E., McClay, E., Bethel, K., Ferrone, S., Hicks, J., & Kuhn, P. (2015). Limited genomic heterogeneity of circulating melanoma cells in advanced stage patients. Physical biology, 12(1), 016008. doi: 10.1088/1478-3975/12/1/016008
51. Welter, L., Xu, L., McKinley, D., Dago, A. E., Prabakar, R. K., Restrepo-Vassalli, S., Xu, K., Rodriguez-Lee, M., Kolatkar, A., Nevarez, R., Ruiz, C., Nieva, J., Kuhn, P., & Hicks, J. (2020). Treatment response and tumor evolution: lessons from an extended series of multianalyte liquid biopsies in a metastatic breast cancer patient. Cold Spring Harbor molecular case studies, 6(6), a005819. doi: 10.1 101/mcs.a005819
52. Malihi, P. D., Morikado, M., Welter, L., Liu, S. T., Miller, E. T., Cadaneanu, R. M., Knudsen, B. S., Lewis, M. S., Carlsson, A., Velasco, C. R., Kolatkar, A., Rodriguez-Lee, M., Garraway, I. P., Hicks, J., & Kuhn, P. (2018). Clonal diversity revealed by morphoproteomic and copy number profiles of single prostate cancer cells at diagnosis. Convergent science physical oncology, 4(1), 015003. doi: 10.1088/2057-1739/aaa00b
53. National Comprehensive Cancer Network. (2021). Breast cancer (version 8.2021). Retrieved from https://www.nccn.org/guidelines/guidelines-detail?category=1 &id=1419
54. Saginala, K.; Barsouk, A.; Aluru, J. S.; Rawla, P.; Padala, S. A.; Barsouk, A. Epidemiology of Bladder Cancer. Med Sci (Basel) 596 2020, 8, 15, doi:10.3390/medsci8010015. 597
55. Hansel, D. E.; Amin, M. B.; Comperat, E.; Cote, R. J.; Knuchel, R.; Montironi, R.; Reuter, V. E.; Soloway, M. S.; Umar, S. A.; Van 598 der Kwast, T. H. A contemporary update on pathology standards for bladder cancer: transurethral resection and radical 599 cystectomy specimens. Eur Urol 2013, 63, 321-332, doi:10.1016/j.eururo.2012.10.008. 600
56. Hasnain, Z.; Mason, J.; Gill, K.; Miranda, G.; Gill, L S.; Kuhn, P.; Newton, P. K. Machine learning models for predicting post-601 cystectomy recurrence and survival in bladder cancer patients. PLoS One 2019, 14, e0210976, 602 doi:10.1371/journal.pone.0210976. 603
57. Mason, J.; Hasnain, Z.; Miranda, G.; Gill, K.; Djaladat, H.; Desai, M.; Newton, P. K.; Gill, L S.; Kuhn, P. Prediction of Metastatic 604 Patterns in Bladder Cancer: Spatiotemporal Progression and Development of a Novel, Web-based Platform for Clinical 605 Utility. European Urology Open Science 2021, 32, 8-18, doi:https://doi.org/10.1016/j.euros.2021.07.006. 606 Cancers 2021, 13, x FOR PEER REVIEW 17 of 19
58. Stein, J. P.; Lieskovsky, G.; Cote, R.; Groshen, S.; Feng, A C.; Boyd, S.; Skinner, E.; Bochner, B.; Thangathurai, D.; Mikhail, M.; 607 et al. Radical cystectomy in the treatment of invasive bladder cancer: long-term results in 1,054 patients. J Clin Oneal 2001, 608 19, 666-675, doi:10.1200/jco.2001.19.3.666. 609
59. Svatek, R. S.; Shariat, S. F.; Novara, G.; Skinner, E. C.; Fradet, Y.; Bastian, P. J.; Kamat, A M.; Kassouf, W.; Karakiewicz, P. I.; 610 Fritsche, H. M.; et al. Discrepancy between clinical and pathological stage: external validation of the impact on prognosis in 611 an international radical cystectomy cohort. BJU Int 2011, 107, 898-904, doi:10.1111/j.1464-410X.2010.09628.x. 612
60. Blick, C. G.; Nazir, S. A; Mallett, S.; Tumey, B. W.; Onwu, N. N.; Roberts, L S.; Crew, J. P.; Cowan, N. C. Evaluation of diagnostic 613 strategies for bladder cancer using computed tomography (CT) urography, flexible cystoscopy and voided urine cytology: 614 results for 778 patients from a hospital haematuria clinic. BJU Int 2012, 110, 84-94, doi:10.1111/j.1464-410X.2011.10664.x. 615
61. Lodewijk, I.; Duenas, M.; Rubio, C.; Munera-Maravilla, E.; Segovia, C.; Bernardini, A; Teijeira, A; Paramio, J. M.; Suarez-616 Cabrera, C. Liquid Biopsy Biomarkers in Bladder Cancer: A Current Need for Patient Diagnosis and Monitoring. Int J Mal 617 Sci 2018, 19, doi:10.3390/ijms19092514. 618
62. Busetto, G. M.; Ferro, M.; Del Giudice, F.; Antonini, G.; Chung, B.1.; Sperduti, I.; Giannarelli, D.; Lucarelli, G.; Borghesi, M.; 619 Musi, G.; et al. The Prognostic Role of Circulating Tumor Cells (CTC) in High-risk Non-muscle-invasive Bladder Cancer. 620 Clin Genitourin Cancer 2017, 15, e661-e666, doi:10.1016/j.clgc.2017.01.011. 621
63. Zhang, Z.; Fan, W.; Deng, Q.; Tang, S.; Wang, P.; Xu, P.; Wang, J.; Yu, M. The prognostic and diagnostic value of circulating 622 tumor cells in bladder cancer and upper tract urothelial carcinoma: a meta-analysis of 30 published studies. Oncotarget 2017, 623 8, 59527-59538, doi:10.18632/oncotarget.18521. 624
64. Pantel, K.; Alix-Panabieres, C.; Riethdorf, S. Cancer micrometastases. Nature Reviews Clinical Oncology 2009, 6, 339-351, 625 doi:10.1038/nrclinonc.2009.44. 626
65. Chai, S.; Matsumoto, N.; Storgard, R.; Peng, C.-C.; Aparicio, A; Ormseth, B.; Rappard, K.; Cunningham, K.; Kolatkar, A; 627 Nevarez, R.; et al. Platelet-coated circulating tumor cells are a predictive biomarker in patients with metastatic castrate 628 resistant prostate cancer. Molecular Cancer Research 2021, molcanres.MCR-21-0383-A2021, doi:10.1158/1541-7786.Mcr-21-629 0383. 630
66. Malihi, P. D.; Graf, R. P.; Rodriguez, A; Ramesh, N.; Lee, J.; Sutton, R.; Jiles, R.; Ruiz Velasco, C.; Sei, E.; Kolatkar, A; et al. 631 Single-Cell Circulating Tumor Cell Analysis Reveals Genomic Instability as a Distinctive Feature of Aggressive Prostate 632 Cancer. Clin Cancer Res 2020, doi:10.1158/1078-0432.CCR-19-4100. 633
67. Malihi, P. D.; Morikado, M.; Welter, L.; Liu, S. T.; Miller, E. T.; Cadaneanu, R. M.; Knudsen, B. S.; Lewis, M. S.; Carlsson, A; 634 Velasco, C. R.; et al. Clonal diversity revealed by morphoproteomic and copy number profiles of single prostate cancer cells 635 at diagnosis. Convergent science physical oncology 2018, 4, doi:10.1088/2057-1739/aaa00b. 636
68. Kuhn, P.; Keating, S. M.; Baxter, G. T.; Thomas, K.; Kolatkar, A; Sigman, C. C. Lessons Learned: Transfer of the High-637 Definition Circulating Tumor Cell Assay Platform to Development as a Commercialized Clinical Assay Platform. Clin 638 Pharmacol Ther 2017, 102, 777-785, doi:10.1002/cpt.645. 639
69. Rodriguez-Lee, M.; Kolatkar, A; McCormick, M.; Dago, A D.; Kendall, J.; Carlsson, N. A; Bethel, K.; Greenspan, E. J.; Hwang, 640 S. E.; Waitman, K. R.; et al. Effect of Blood Collection Tube Type and Time to Processing on the Enumeration and High-641 Content Characterization of Circulating Tumor Cells Using the High-Definition Single-Cell Assay. Arch Pathol Lab Med 2018, 642 142, 198-207, doi:10.5858/arpa.2016-0483-OA 643
70. Shishido, S. N.; Welter, L.; Rodriguez-Lee, M.; Kolatkar, A; Xu, L.; Ruiz, C.; Gerdtsson, A S.; Restrepo-Vassalli, S.; Carlsson, 644 A; Larsen, J.; et al. Preanalytical Variables for the Genomic Assessment of the Cellular and Acellular Fractions of the Liquid 645 Biopsy in a Cohort of Breast Cancer Patients. J Mal Diagn 2020, 22, 319-337, doi:10.1016/j.jmoldx.2019.11.006. 646 Cancers 2021, 13, x FOR PEER REVIEW 18 of 19
71. Marrinucci, D.; Bethel, K.; Kolatkar, A.; Luttgen, M. S.; Malchiodi, M.; Baehring, F.; Voigt, K.; Lazar, D.; Nieva, J.; Bazhenova, 647 L.; et al. Fluid biopsy in patients with metastatic prostate, pancreatic and breast cancers. Phys Biol 2012, 9, 016003, 648 doi: 10.1088/1478-3975/9/1/016003. 649
72. Carlsson, A.; Kuhn, P.; Luttgen, M. S.; Dizon, K. K.; Troncoso, P.; Com, P. G.; Kolatkar, A.; Hicks, J. B.; Logothetis, C. J.; Zurita, 650 A. J. Paired High-Content Analysis of Prostate Cancer Cells in Bone Marrow and Blood Characterizes Increased Androgen 651 Receptor Expression in Tumor Cell Clusters. Clin Cancer Res 2017, 23, 1722-1732, doi:10.1158/1078-0432.Ccr-16-1355. 652
73. Gerdtsson, A. S.; Setayesh, S. M.; Malihi, P. D.; Ruiz, C.; Carlsson, A.; Nevarez, R.; Matsumoto, N.; Gerdtsson, E.; Zurita, A.; 653 Logothetis, C.; et al. Large Extracellular Vesicle Characterization and Association with Circulating Tumor Cells in Metastatic 654 Castrate Resistant Prostate Cancer. Cancers (Basel) 2021, 13, doi:10.3390/cancers13051056. 655
74. Ruiz, C.; Li, J.; Luttgen, M. S.; Kolatkar, A.; Kendall, J. T.; Flores, E.; Topp, Z.; Samlowski, W. E.; McClay, E.; Bethel, K.; et al. 656 Limited genomic heterogeneity of circulating melanoma cells in advanced stage patients. Phys Biol 2015, 12, 016008, 657 doi:10.1088/1478-3975/12/1/016008. 658
75. Spearman, C. The proof and measurement of association between two things. By C. Spearman, 1904. Am J Psychol 1987, 100, 659 441-471. 660
76. Mann, H. B.; Whitney, D. R. On a Test of Whether one of Two Random Variables is Stochastically Larger than the Other. The 661 Annals of Mathematical Statistics 1947, 18, 50-60, 11. 662
77. Wilcoxon, F. Individual comparisons of grouped data by ranking methods. J Econ Entomol 1946, 39, 269, 663 doi:10.1093/jee/39.2.269. 664
78. McIntosh, A. M.; Sharpe, M.; Lawrie, S. M. 9—Research methods, statistics and evidence-based practice. In Companion to 665 Psychiatric Studies (Eighth Edition), Johnstone, E. C., Owens, D. C., Lawrie, S. M., McIntosh, A. M., Sharpe, M., Eds.; Churchill 666 Livingstone: St. Louis, 2010; pp. 157-198. 667
79. Maaten, L. v. d.; Hinton, G. E. Visualizing Data using t-SNE. Journal of Machine Learning Research 2008, 9, 2579-2605. 668
80. Pedregosa, F.; Varoquaux, G.; Gramfort, A.; Michel, V.; Thirion, B.; Grisel, 0.; Blonde!, M.; Louppe, G.; Prettenhofer, P.; 669 Weiss, R.; et al. Scikit-leam: Machine Learning in Python. J. Mach. Learn. Res. 2011, 12, 2825-2830. 670
81. Ward, J. H. Hierarchical Grouping to Optimize an Objective Function. Journal of the American Statistical Association 1963, 58, 671 236-244, doi:10.1080/01621459.1963.10500845. 672
82. Zhu, C. Z.; Ting, H. N.; Ng, K. H.; Ong, T. A. A review on the accuracy of bladder cancer detection methods. J Cancer 2019, 10, 673 4038-4044, doi:10.7150/jca.28989. 674
83. Yafi, F. A.; Brimo, F.; Steinberg, J.; Aprikian, A. G.; Tanguay, S.; Kassouf, W. Prospective analysis of sensitivity and specificity 675 of urinary cytology and other urinary biomarkers for bladder cancer. Urol Oncol 2015, 33, 66.e25-31, 676 doi:10.1016/j.urolonc.2014.06.008. 677
84. Gazzaniga, P.; Gradilone, A.; de Berardinis, E.; Busetto, G. M.; Raimondi, C.; Gandini, O.; Nicolazzo, C.; Petracca, A.; 678 Vincenzi, B.; Farcomeni, A.; et al. Prognostic value of circulating tumor cells in nonmuscle invasive bladder cancer: a 679 CellSearch analysis. Annals of Oncology 2012, 23, 2352-2356, doi:10.1093/annonc/mdr619. 680
85. Bethel, K.; Luttgen, M. S.; Damani, S.; Kolatkar, A.; Lamy, R.; Sabouri-Ghomi, M.; Topol, S.; Topol, E. J.; Kuhn, P. Fluid phase 681 biopsy for detection and characterization of circulating endothelial cells in myocardial infarction. Phys Biol 2014, 11, 016002, 682 doi:10.1088/1478-3975/ll/l/016002. 683
86. Liang, L. G.; Kong, M. Q.; Zhou, S.; Sheng, Y. F.; Wang, P.; Yu, T.; Ind, F.; Kuo, W. P.; Li, L. J.; Demirci, U.; et al. An integrated 684 double-filtration microfluidic device for isolation, enrichment and quantification of urinary extracellular vesicles for 685 detection of bladder cancer. Sci Rep 2017, 7, 46224, doi:10.1038/srep46224. 686
87. Armstrong, A. J.; Halabi, S.; Luo, J.; Nanus, D. M.; Giannakakou, P.; Szmulewitz, R. Z.; Danila, D. C.; Healy, P.; Anand, M.; 687 Rothwell, C. J.; et al. Prospective Multicenter Validation of Androgen Receptor Splice Variant 7 and Hormone Therapy 688 Cancers 2021, 13, x FOR PEER REVIEW 19 of 19 Resistance in High-Risk Castration-Resistant Prostate Cancer: The PROPHECY Study. J Clin Oneal 2019, 37, 1120-1129, 689 doi:10.1200/jco.18.01731. 690
88. Dago, A. E.; Stepansky, A.; Carlsson, A.; Luttgen, M.; Kendall, J.; Baslan, T.; Kolatkar, A.; Wigler, M.; Bethel, K; Gross, M. E.; 691 et al. Rapid phenotypic and genomic change in response to therapeutic pressure in prostate cancer inferred by high content 692 analysis of single circulating tumor cells. PLaS One 2014, 9, e101777, doi:10.1371/joumal.pone.0101777. 693
89. Scher, H. I.; Graf, R. P.; Schreiber, N. A.; Winquist, E.; McLaughlin, B.; Lu, D.; Orr, S.; Fleisher, M.; Lowes, L.; Anderson, A. K L.; 694 et al. Validation of nuclear-localized AR-V7 on circulating tumor cells (CTC) as a treatment-selection biomarker for 695 managing metastatic castration-resistant prostate cancer (mCRPC). J Clin Oneal 2018, 36, doi:DOI 696 10.1200/JCO.2018.36.6_suppl.273. 697
90. Scher, H. I.; Lu, D.; Schreiber, N. A.; Louw, J.; Graf, R. P.; Vargas, H. A.; Johnson, A.; Jendrisak, A.; Bambury, R.; Danila, D.; et 698 al. Association of AR-V7 on Circulating Tumor Cells as a Treatment-Specific Biomarker With Outcomes and Survival in 699 Castration-Resistant Prostate Cancer. JAMA Oneal 2016, 2, 1441-1449, doi:10.1001/jamaoncol.2016.1828. 700
91. Thiele, J. A.; Bethel, K; Kralickova, M.; Kuhn, P. Circulating Tumor Cells: Fluid Surrogates of Solid Tumors. Annu Rev Pathal 701 2017, 12, 419-447, doi:10.1146/annurev-pathol-052016-100256. 702
92. Birkenkamp-Demtroder, K; Christensen, E.; Nordentoft, I.; Knudsen, M.; Taber, A.; Hoyer, S.; Lamy, P.; Agerbaek, M.; 703 Jensen, J. B.; Dyrskjot, L. Monitoring Treatment Response and Metastatic Relapse in Advanced Bladder Cancer by Liquid 704 Biopsy Analysis. Eur Ural 2018, 73, 535-540, doi:10.1016/j.eururo.2017.09.011. 705
93. Birkenkamp-Demtroder, K; Nordentoft, I.; Christensen, E.; Hoyer, S.; Reinert, T.; Vang, S.; Borre, M.; Agerbaek, M.; Jensen, 706 J. B.; Omtoft, T. F.; et al. Genomic Alterations in Liquid Biopsies from Patients with Bladder Cancer. Eur Ural 2016, 70, 75-82, 707 doi:10.1016/j.eururo.2016.01.007. 708
94. Christensen, E.; Birkenkamp-Demtroder, K; Nordentoft, I.; Hoyer, S.; van der Kern, K; van Kessel, K; Zwarthoff, E.; 709 Agerbaek, M.; Omtoft, T. F.; Jensen, J. B.; et al. Liquid Biopsy Analysis of FGFR3 and PIK3CA Hotspot Mutations for Disease 710 Surveillance in Bladder Cancer. Eur Ural 2017, 71, 961-969, doi:10.1016/j.eururo.2016.12.016. 711
95. Patel, K M.; van der Vos, K E.; Smith, C. G.; Mouliere, F.; Tsui, D.; Morris, J.; Chandrananda, D.; Marass, F.; van den Broek, 712 D.; Neal, D. E.; et al. Association Of Plasma And Urinary Mutant DNA With Clinical Outcomes In Muscle Invasive Bladder 713 Cancer. Sci Rep 2017, 7, 5554, doi:10.1038/s41598-017-05623-3. 714
96. Chalfin, H. J.; Glavaris, S. A.; Gorin, M. A.; Kates, M. R.; Fong, M. H.; Dong, L.; Matoso, A.; Bivalacqua, T. J.; Johnson, M. H.; 715 Pienta, K J.; et al. Circulating Tumor Cell and Circulating Tumor DNA Assays Reveal Complementary Information for 716 Patients with Metastatic Urothelial Cancer. Eur Ural Oneal 2021, 4, 310-314, doi:10.1016/j.euo.2019.08.004. 717 718

1. A biological structure identification system comprising:
an optical imaging system configured to illuminate a liquid biopsy sample for a subject, the liquid biopsy sample having one or more biological structures that are labeled with one or more fluorophores associated with a fluorescence assay for a cancer allowing detection of emitted electromagnetic radiation from the liquid biopsy sample as image data; and
a processing system configured to:
generate images of the one or more biological structures for the subject from the image data, detect and determine a plurality of features from the images or the image data, and form biological structure identification buckets from the plurality of features, each biological structure identification bucket identifying biological structures that are similar in type;
generating a subject profile of biological structure identification buckets for rare biological structures for the subject;
comparing the subject profile with a set of predetermined cancer stage profiles of subjects having the cancer at a plurality of cancer stages; and
identifying a cancer stage for the subject by determining a predetermined cancer stage profile from the set of predetermined cancer stage profiles to which the subject profile is most similar.

2. The biological structure identification system of claim 1, wherein the rare biological structures are observed as rare imaging events in the imaging data.

3. The biological structure identification system of claim 1, wherein the one or more biological structures include simultaneously identified multiple biological structures.

4. The biological structure identification system of claim 1, wherein the optical imaging system includes:
a liquid biopsy sample carrier suitable for receiving supporting the liquid biopsy sample;
an illumination system capable of illuminating the liquid biopsy sample at a specific wavelength or wavelengths that can be absorbed by the one or more fluorophores;
a light detection system configured to detect and determine an intensity and a wavelength of fluorescence emitted by the one or more fluorophores; and
a light controlling system configured to allow detection of emitted electromagnetic radiation from the liquid biopsy sample; allow detection of electromagnetic radiation scattered by, reflected by, and/or transmitted through the liquid biopsy sample; and guide electromagnetic radiation from the illumination system to the liquid biopsy sample, and from the liquid biopsy sample to the light detection system.

5. The biological structure identification system of claim 1, wherein the processing system configured to:
detect and determine morphology of each biological structure using each image;
identify type of each biological structure from the plurality of features;
form the biological structure identification bucket plurality of features; and
form a set of identification buckets based on identification buckets.

6. The biological structure identification system of claim 1 wherein at least a subset of the one or more biological structures includes a component selected from the group consisting of a structure with a membrane, a protein, DNA, RNA, and combinations thereof.

7. The biological structure identification system of claim 1 wherein at least a subset of the one or more biological structures is a structure with a membrane selected from the group consisting of a cell, a vesicle, and combinations thereof.

8. The biological structure identification system of claim 7 wherein the vesicle is an oncosome.

9. The biological structure identification system of claim 7 wherein the vesicle is an oncosome that has a characteristic size equal to or larger than one micrometer.

10. The biological structure identification system of claim 7 wherein the vesicle is an oncosome that has a characteristic size larger than exosome.

11. The biological structure identification system of claim 1 wherein the liquid biopsy sample is a non-solid biological sample.

12. The biological structure identification system of claim 1 wherein the liquid biopsy sample is a body fluid sample.

13. The biological structure identification system of claim 1 wherein the liquid biopsy sample comprises a blood sample, a bone marrow sample, a peritoneal fluid sample, a urine sample, a saliva, a vaginal fluid sample, a semen sample, a tear sample, a mucus sample, an aqueous humor sample, cerebrospinal fluid (CSF) sample, or a combination thereof.

14. The biological structure identification system of claim 1 wherein the liquid biopsy sample comprises a blood sample.

15. The biological structure identification system of claim 1 wherein the liquid biopsy sample comprises common immune cells and rare biological structures.

16. The biological structure identification system of claim 15 wherein the rare biological structures are:
cancer cells that have cancer genomic profiles and/or cancer protein markers;
tumor microenvironment cells that leak into circulation, wherein these cells comprise epithelial cells, endothelial cells, mesenchymal cells, other stromal cells, cells that are in various transitional states, or a mixture thereof;
immune cells that are responding to a tumor itself or cancer treatment;
vesicles; or
mixtures thereof.

17. The biological structure identification system of claim 15 wherein the rare biological structures comprise:
conventional circulating tumor cells, which are CK+, vimentin−, CD31− and CD45−; circulating tumor cells, which are CK+, CD31−, CD45−, and vimentin+, and wherein tumor cells may putatively in epithelial to mesenchymal transition;
tumor cells, which are CK+, and coated with platelets, which are CD31+;
endothelial cells, which are CD31+, vimentin+, and CK−;
endothelial cells, which are CD31+, vimentin+ and CK+;
megakaryocytes, which are CD31+ and vimentin−, wherein megakaryocytes include large cells containing a single, large, multi-lobulated, polyploidy nucleus responsible for production of blood thrombocytes platelets;
large cells, which are CD31+ and CK+, wherein these large cells may be present in liquid biopsy samples obtained from a bone marrow;
cells, which are DAPI+ and vimentin+;
round cells, which are CD45+ and CK+;
round cells, which are CD45+, vimentin+, and CK+;
clusters of cells (“cell cluster’) comprising at least two cells, wherein the cells are same type of cells and/or different types of cells;
cells, which are DAPI+, CD45−, CD31−, and CK−;
immune cells, which are CD45+ and vimentin−;
immune cells, which are CD45+ and vimentin+ (type III intermediate filament protein), extra-cellular vesicles; or
a mixture thereof.

18. The biological structure identification system of claim 1, wherein the liquid biopsy sample comprises common biological structures and rare biological structures such that a total number of biological structures is a sum of the number of common biological structures and rare biological structures, and wherein the rare biological structures are present in an amount equal to or less than 10%, 5%, 1%, 0.1%, or 0.01% of the total number of biological structures.

19. The biological structure identification system of claim 1, wherein the emitted electromagnetic radiation is a fluorescent radiation.

20. The biological structure identification system of claim 1 wherein, wherein the optical imaging system comprises an excitation filter, an emission filter, a (dichroic) mirror, a lens, an optical fiber, or a combination thereof.

21. The biological structure identification system of claim 1 wherein, wherein the optical imaging system comprises a fluorescence microscope, a brightfield microscope, or a combination thereof.

22. The biological structure identification system of claim 1 wherein, wherein the processing system comprises a control system, a hardware processor, a memory system, and an information conveying system.

23. The biological structure identification system of claim 1, wherein the biological structure identification system has at least one fluorescence channel.

24. The biological structure identification system of claim 21, wherein the biological structure identification system applies 1 to 10 fluorescence channels.

25. The biological structure identification system of claim 21 wherein the biological structure identification system has 4 to 7 fluorescence channels.

26. The biological structure identification system of claim 1, wherein the biological structure identification system has only four fluorescence channels.

27. The biological structure identification system of claim 1, wherein the biological structure identification system has only four fluorescence channels, wherein the four fluorescence channels are:
a first fluorescence channel configured for detection useful for nuclear segmentation and characterization;
a second fluorescence channel configured to detect a cytokeratin (CK) for its epithelial-like phenotype;
a third fluorescence channel configured to detect a vimentin for its endothelial/mesenchymal-like phenotype; and
a fourth fluorescence channel configured to detect both a CD31 for its endothelial-like phenotype, and a CD45 for its immune cell phenotype.

28. The biological structure identification system of claim 1, wherein the biological structure identification system has only four fluorescence channels, wherein the four fluorescence channels are:
a first fluorescence channel configured for detection of fluorescence emission at a blue color wavelength region;
a second fluorescence channel configured for detection of fluorescence emission at a red color wavelength region;
a third fluorescence channel configured for detection of fluorescence emission at an orange color wavelength region; and
a fourth fluorescence channel configured for detection of fluorescence emission at a green color wavelength region.

29. The biological structure identification system of claim 28, wherein the first fluorescence channel is configured to detect 4′,6-diamidino-2-phenylindole (DAPI) for nuclear segmentation and characterization.

30. The biological structure identification system of claim 28, wherein the biological structure identification system has only four fluorescence channels and is configured to identify endothelial cells and immune cells from the plurality of features.

31. The biological structure identification system of claim 30, wherein the biological structure identification system has only four fluorescence channels and is configured to identify the endothelial cells and the immune cells from the plurality of features, and to differentiate the endothelial cells from the immune cells, wherein the endothelial cells have more elongated morphologies as compared to the immune cells, and the immune cells have more round morphologies as compared to the endothelial cells.

32. The biological structure identification system of claim 1, wherein a biological structure's morphology is determined by using at least one feature from the images or the image data.

33. The biological structure identification system of claim 1, wherein a biological structure's morphology is determined from at least 10 features, at least 100 features, at least 500 features, or at least 1,000 features from the images or the image data.

34. The biological structure identification system of claim 33, wherein at least a subset of the features are related to size, shape, texture and structure of the one or more biological structures.

35. The biological structure identification system of claim 1, wherein the liquid biopsy sample is obtained from a diseased human.

36. The biological structure identification system of claim 1, wherein the liquid biopsy sample is obtained from a human afflicted with the cancer.

37. The biological structure identification system of claim 1, wherein processing system is further configured to form a disease map based on information related to the biological structure identification bucket set(s), relate the disease map to a specific disease and disease stage, and label the disease map according to an identified related specific disease and disease stage.

38. The biological structure identification system of claim 1, wherein the biological structure identification system is further configured to store a disease map based on information related to one or more identification bucket sets and labeled by disease type and disease stage, and wherein a disease afflicting a subject causes formation of the biological structures forming the one or more identification bucket sets.

39. The biological structure identification system of claim 1, wherein the biological structure identification system is further configured to diagnose a disease type and its stage based on a received liquid biopsy sample from a human afflicted with a disease.

40. The biological structure identification system of claim 1, wherein the biological structure identification system further comprises an information conveying system that is configured to convey to a user information related to types of the biological structures present in the liquid biopsy sample, biological structure identification buckets, disease maps, disease atlases, or a combination thereof.

41. A method of diagnosing a disease with the biological structure identification system of claim 1, the method comprising:
receiving a liquid biopsy sample from a subject comprising biological structures;
preparing a sample comprising a single biological structure layer sample from the liquid biopsy sample, the single biological structure layer sample being a single layer of biological structures;
staining the biological structures of the single layer biological structure sample with a fluorescence assay for a cancer; and
analyzing the sample with a biological structure identification system configured to identify rare biological structures through their fluorescence and morphology and form one or more biological structure identification buckets based on an identified biological structure type, wherein each biological structure identification bucket contains a similar type of biological structures;
generating a subject profile of biological structure identification buckets for rare biological structures for the subject;
comparing the subject profile with a set of predetermined cancer stage profiles of subjects having the cancer at a plurality of cancer stages; and
identifying a cancer stage for the subject by determining a predetermined cancer stage profile from the set of predetermined cancer stage profiles to which the subject profile is most similar.
